(19) United States
(12) Patent Application Publication
(10) Pub. No.: US 2021/0283059 A1
(43) Pub. Date: Sep. 16, 2021

(54) HYDROPHOBICITY/HYDROPHILICITY-TUNABLE ORGANOSILOXANE NANO-/MICROSPHERES AND PROCESS TO MAKE THEM

(71) Applicant: SILICYCLE INC., Quebéc (CA)

(72) Inventors: Lilit Aboshyan-Sorgho, Quebéc (CA); François Beland, Quebéc (CA); Meryem Bouchoucha, Quebéc (CA); Delphine Desplantier-Giscard, Quebéc (CA); Simon Giret, Quebéc (CA); Michel Morin, Quebéc (CA); Valerica Pandarus, Quebéc (CA); Xiaowei Wu, Quebéc (CA)

(21) Appl. No.: 17/290,052

(22) PCT Filed: Nov. 12, 2019
(86) PCT No.: PCT/CA2019/051605

§ 371 Date: Apr. 29, 2021

Related U.S. Application Data
(60) Provisional application No. 62/760,413, filed on Nov. 13, 2018.

(51) Int. Cl. A61K 9/16; A61K 31/513

(52) U.S. Cl. CPC A61K 9/1682; B82Y 5/00; A61K 31/513; A61K 9/1641

(57) ABSTRACT

The present disclosure relates to hydrophobicity/hydrophilicity-tunable organosiloxane nano-/microspheres with or without actives/payloads and a one-pot surfactant-free versatile process to make them. The release can be controlled by adjusting the hydrophobicity/hydrophilicity of the organosiloxane nano-/microspheres. The process of preparation comprising i0) separately hydrolyzing one or more silica precursor in a hydrolytic media; i1) combining the pre-hydrolyzed precursors or i2) removing a part of or totality of volatile solvents or i3) preparing a dispersed phase comprising a hydrophilic solvent to provide a dispersed phase; emulsifying, in absence of a surfactant, the dispersed phase of the step i1), i2) or i3) in a continuous phase to provide a water in oil emulsion; i5) adding a condensation catalyst to the emulsion to provide said nano-/microspheres.

### FIELD OF THE DISCLOSURE

The present disclosure relates to hydrophobicity/hydrophilicity-tunable organosiloxane nano-/microspheres with or without actives/payloads and a process to make them. The release can be controlled by adjusting the hydrophobicity/hydrophilicity of the organosiloxane nano-/microspheres.

### BACKGROUND

The actives/payloads sequestration has been widely adapted in the last couple of decades in an increasing number of industrial sectors for different purposes (e.g. pharmaceutic, cosmetic, food, construction, agriculture, catalysis) owing to a number of accompanying attractive properties of this technique, such as reducing volatility, shielding unpleasant odors, protecting unstable payloads, preventing premature release of active materials, achieving better handling and the better controlling of the payload liberation.

Organosiloxane materials are particularly interesting due to their intrinsic advantages, such as chemical inertness, mechanical robustness, controllable morphology, adjustable porosity and versatile functions. Furthermore, organosiloxane materials have been considered as GRAS (i.e. Generally Recognized As Safe).

### SUMMARY OF THE DISCLOSURE

In one aspect, there is provided a process of preparation of organosiloxane nano-/microspheres comprising:

i0) separately hydrolyzing one or more silica precursor in a hydrolytic media to provide one or more pre-hydrolyzed silica precursor;
i1) combining the pre-hydrolyzed silica precursors of step iG) to provide a dispersed phase comprising combined pre-hydrolyzed silica precursors; or
i2) removing a part or totality of volatile solvents from said combined pre-hydrolyzed silica precursors to provide a dispersed phase comprising pre-condensed silica precursors; or
i3) preparing a dispersed phase comprising a hydrophilic solvent by adding said hydrophilic solvent to said dispersed phase comprising combined pre-hydrolyzed silica precursors obtained in step i1) or by adding said hydrophilic solvent to said dispersed phase comprising pre-condensed silica precursors obtained in step i2);
i4) emulsifying, in absence of a surfactant, the dispersed phase of the step i1), i2) or i3) in a continuous phase to provide a water in oil emulsion;
i5) adding a condensation catalyst to the emulsion of step i4) to provide said organosiloxane nano-/microspheres.

In a further aspect, there is provided an organosiloxane spheroidal nano-/microspheres comprising a network consisting of organo-siloxane, wherein said particle is uncalcined amorphous, surfactant-free and is sub-micron to micron size, particle optionally comprising an active/payload.

In still a further aspect, there is provided a method for modulating the release of an active/payload, comprising incorporating said active/payload in nano-/microspheres as defined herein, or incorporating said active/payload in a process as defined herein.

### BRIEF DESCRIPTION OF THE FIGURES

The illustrations of the examples corresponding figures are listed as bellow:

FIG. 1. SEM images of the examples of microspheres: A) Example 1-1(scale bar=200 μm), B) Example 1-2 (scale bar=200 μm) and C) Example 1-3 (scale bar=10 μm).

FIG. 2. SEM images of the examples of microspheres: A) Example 2-1 (scale bar=200 μm) and B) Example 2-2 (scale bar=10 μm).

FIG. 3. SEM images of examples of the obtained microspheres. A) Example 3 (scale bar=40 μm), B) Example 4 (scale bar=200 μm), C) Example 5 (scale bar=100 μm), D) Example 6-1 (scale bar=5 μm), E) Example 6-2 (scale bar=10 μm), F) Example 7 (scale bar=10 μm), G) Example 8 (scale bar=10 μm), H) Example 9 (scale bar=100 μm), I) Example 10 (scale bar=3 μm), J) Example 11 (scale bar=400 μm), K) Example 12 (scale bar=5 μm), L) Example 13 (scale bar=100 μm), M) Example 14 (scale bar=200 μm), N) Example 15 (scale bar=500 μm), 0) Example 16 (scale bar=100 μm), P) Example 17-1 (scale bar=200 μm), Q) Example 17-2 (scale bar=100 μm), R) Example 17-3 (scale bar=100 μm), S) Example 17-4 (scale bar=100 μm) and T) Example 18 (scale bar=100 μm).

FIG. 4. Contact angle pictures. A) Example 15, B) Example 4 and C) Example 5.

FIG. 5. SEM images of the examples of microspheres containing active/payload. A) Example 20-1 (scale bar=200 μm), B) Example 20-2 (scale bar=100 μm) and C) Example 20-3 (scale bar=70 μm).

FIG. 6. SEM images of the examples of microspheres containing active/payload. A) Example 21-1 (scale bar=10 μm), B) Example 21-2 (scale bar=100 μm), C) Example 21-3 (scale bar=100 μm) and D) Example 21-4 (scale bar=100 μm).

FIG. 7. SEM images of the examples of microspheres containing active/payload. A) Example 22-1 (scale bar=100 μm), B) Example 22-2 (scale bar=10 μm) and C) Example 22-3 (scale bar=10 μm).

FIG. 8. SEM images of the examples of microspheres charged with active/payload. A) Example 23-1 (scale bar=100 μm) and B) Example 23-2 (scale bar=10 μm).

FIG. 9. SEM images of the examples of microspheres charged with active/payload. A) Example 24-1 (scale bar=10 μm), B) Example 24-2 (scale bar=500 μm), c) Example 24-3 (scale bar=100 μm), D) Example 24-4 (scale bar=10 μm), E) Example 24-5 (scale bar=50 μm), F) Example 24-6 (scale bar=50 μm), G) Example 24-7 (scale bar=50 μm), H) Example 24-8 (scale bar=50 μm), I) Example 24-9 (scale bar=100 μm), J) Example 24-10 (scale bar=100 μm) and K) Example 25 (scale bar=120 μm).

FIG. 10. Uracil release profiles from the developed microspheres in pH 7 (A, C, and D) and in pH 5.5 (B)

### DETAILED DESCRIPTION

The present disclosure relates to a versatile process. This process is providing 1) a one pot process, 2) with or without in-situ actives/payloads administration/sequestration method to distribute the active ingredients throughout the organosiloxane spherical materials in solid state or liquid state, 3) adjustable hydrophobic/hydrophilic property of pre-hydrolyzed/pre-condensed silica precursors to be compatible with active ingredients and 4) controlling the actives/payloads release parameters by tailoring the hydrophobicity and hydrophilicity of the external and internal surface of the organosiloxane spherical materials.

The process herein is conducted without a surfactant. Surfactants exhibit a series of disadvantages due to the required additional washing steps and potential residual contamination left in the organosiloxane nano-/microspheres. The use of surfactants therefore entails supplementary costs/production time.

A surfactant is understood of any such agent not taking part in the siloxane network (forming Si—O—Si) bonds. Certain silica precursors used herein may have amphiphilic parts but are however not excluded from the process herein as they participate in creating the siloxane network.

The process and organosiloxane nano-/microspheres are free of surfactant other than amphiphilic silanes.

The process herein is preferably conducted under high shear or dispersing force.

The “silica precursors” used herein refer to compounds of formula R₄₋ₓ Si(L) x or formula (L)₃Si—R′—Si(L)₃, wherein

R: is mono-silylated residue as an alkyl, alkenyl, alkynyl, alicyclic, aryl, alkyl-aryl group, which is optionally substituted by a halogen atom, —OH, —SH, —N(Rₐ)₂, —N+(Rₐ)₃, —P(Rₐ) ₂;
Rₐ: can be alkyl, alkenyl, alkynyl, alicyclic, aryl and alkyl-aryl;
L: is a halogen or an acetoxide —O—C(O)Rₐ, or alkoxide ORₐ group;
X: is an integer of 1 to 4; and
R′: is bi-silylated residue as an alkyl, alkenyl, alkynyl, alicyclic, aryl, alkyl-aryl group, which is optionally substituted by a halogen atom, —OH, —SH, —N(Rₐ)₂, —N+(Rₐ)₃, —P(Rₐ) 2;
In one embodiment, the silica precursor R₄₋ₓSi(L)ₓ or (L)₃Si—R′—Si(L)₃ is a silicon alkoxide such as tetraalkoxide silane, monoalkyl-trialkoxysilane, or a dialkyl dialkoxysilane or a bis-trialkoxy bridged silane. In a further aspect the silica precursor is a mixture of silicon alkoxides, such as tetraalkoxy silane and/or monoalkyl-trialkoxysilane, and/or dialkyl-dialkoxysilane and/or a bis-trialkoxy bridged silane.

In one embodiment, the monoalkyl trialkoxy silanes RSi(L)₃ comprise monoalkyl, which is linear or branched group of 1 to 18 carbon atoms, and the trialkoxy is triethoxy or trimethoxy group.

In one embodiment, the dialkyl dialkoxy silanes R₂Si(L)₂ comprise dialkyl, which is linear or branched group of 1 to 18 carbon atoms, and the dialkoxy is diethoxy or dimethoxy group.

In one embodiment, the trialkyl monoalkoxy silanes R₃Si(L) comprise trialkyl, which is linear or branched group of 1 to 18 carbon atoms, and the monoalkoxy is monoethoxy or monomethoxy group.

In one embodiment, the trialkoxy bridged silanes (L)₃Si—R′—Si(L)₃ comprise bridged, which is linear alkyl or alkenyl group of 2 to 18 carbon atoms, and the trialkoxy is triethoxy or trimethoxy group.

The hydrolytic media to use in the present disclosure will favor the formation of silanol function Si—OH produced from the hydrolysis of the silica precursors. Examples of such media include aqueous medias, such as water, optionally mixed with a water miscible organic solvent, such as ethanol or THF and an inorganic acid such as HCl, H₃PO₄, H₂SO₄, HNO₃. Preferably the concentration of the hydrolytic media is from about 0.01 mol.l⁻¹ to 0.05 mol.l⁻¹, and preferentially the inorganic acid is HCl.

The condensation catalyst refers to any reagent known in the art to favor the polycondensation to form siloxane Si—O—Si bonds, which achieve the final pH in the suspension at about 9.0 to 11.5. The condensation catalyst can be, but not limited to, NH₄OH, NaOH, KOH, LiOH, Ca(OH)₂, NaF, KF, TBAF, TBAOH, TMAOH, triethanol amine, triethyl amine, primene, L-lysine, aminopropylsilane.

In one embodiment, the condensation catalyst is concentrated NH₄OH. In one embodiment, the condensation catalyst is NaOH.

As used herein, “dispersed phase” means the mixture of the pre-hydrolyzed or/and pre-condensed silica precursors, with or without actives/payloads. Pre-hydrolyzed silica precursors are obtained by the hydrolysis of the L group of R₄₋ₓSi(L)ₓ or (L)₃Si—R′—Si(L)₃ in the hydrolytic media. Pre-condensed silica precursors are obtained by the partial condensation of the pre-hydrolyzed silica precursors by evaporating the volatile solvents present in the hydrolytic media. The disperse phase may also contain one or more hydrophilic solvent.

As used herein, “continuous phase” means solvent known in the art to have opposite polarity compared to the dispersed phase to produce reverse phase emulsion (water in oil).

Continuous phase can be for example but not restricted to toluene, xylene, benzene, hexane, cyclohexane, pentane, heptane, 2-butanone, trichloroethylene, diethyl ether, diisopropyl ether, ethyl acetate, 1,2-dichloromethane, chloroform, carbon tetrachloride, butyl acetate, n-butanol, n-pentanol. In one embodiment, the continuous phase is preferentially toluene, xylene, hexane or cyclohexane.

In one embodiment, the volume ratio of the continuous phase to the dispersed phase containing the pre-hydrolyzed/pre-condensed silica precursors is 5 to 500, preferably 10 to 100.

As used herein, “emulsion process” indicates a process relative to a piece of laboratory or industrial equipment used to mix two or more liquids that are normally immiscible resulting in a dispersion of droplets (dispersed phase) in a volume of continuous phase. Preferably rotor-stator homogenizer and sonic dismembrator.

In one embodiment, rotor-stator homogenizer is used for the emulsion process. Typically, the homogenizer speed is about 4000 rpm to 20000 rpm. Preferably, about 12000 rpm or 20000 rpm.

In one embodiment, sonic dismembrator homogenizer is used for the emulsion process. Typically, the homogenizer power potentiometer is about 50% to 100% with an on/off cycle on from 50% to 100% of the time. Preferably, about 100% for power potentiometer and 100% on for the cycle time.

The size of the microspheres can be modified by the emulsification method. The rotor-stator homogenizer induces the formation of microsphere with an average diameter generally between 1 and 200 μm. The sonic dismembrator induces the formation of nanospheres with an average diameter generally between 0.05 and 10 μm. The size of the nano-/microspheres can be modified by other parameters, such as, the ratio of continuous phase to dispersed phase. The higher the ratio is, the smaller the nano-/microspheres are. The speed of the rotor-stator homogenizer or the power of the sonic dismembrator are important to consider regarding the size of nano-/microspheres. The higher the speed or power is, the smaller the nano-/microspheres are.

As used herein, “actives/payloads” refer to the compounds of interests which will be trapped in the nano-/microspheres. Actives/payloads are preferably insoluble in the continuous phase. The actives/payloads can be in both solid and liquid form. They can be incorporated by solubilization, dispersion or emulsification in the dispersed phase.

In one embodiment, the active/payload is a hydrophilic molecule that can be soluble in aqueous and/or polar solvent.

In one embodiment, the active/payload is a cosmetic, cosmeceutical and pharmaceutical compound.

In one embodiment, uracil is used as active/payload. In one embodiment, 5-fluorouracil is used as active/payload. In one embodiment, said active/payload is a saccharide or a derivative, preferably a mono saccharide such as mannose, (especially D-mannose) and glucose (especially D-glucose). In one embodiment the active is a vitamin (e.g. vitamin C).

In accordance with the disclosure, the general process can involve or not actives/payloads. A) In one embodiment, actives/payloads are not used in any of the process steps. B) In further embodiment, at least one actives/payloads are used during at least one process step.

(Method A) In one embodiment, the process of preparation of silica nano-/microspheres without actives/payloads comprises, A0) separately hydrolyzing one or more silica precursor in a hydrolytic media to provide one or more pre-hydrolyzed silica precursor; A1) combining the pre-hydrolyzed silica precursors of step A0) to provide a dispersed phase comprising combined pre-hydrolyzed silica precursors; or A2) removing a part or totality of volatile solvents from said combined pre-hydrolyzed silica precursors to provide a dispersed phase comprising pre-condensed silica precursors; or A3) preparing a dispersed phase comprising a hydrophilic solvent by adding said hydrophilic solvent to said dispersed phase comprising combined pre-hydrolyzed silica precursors obtained in step A1) or by adding said hydrophilic solvent to said dispersed phase comprising pre-condensed silica precursors obtained in step A2); A4) emulsifying, in absence of a surfactant, the dispersed phase of the step A1), A2) or A3) in the continuous phase to provide a water in oil emulsion; A5) adding a condensation catalyst to the emulsion of step A4) to provide said organosiloxane nano-/microspheres; A6) optionally aging the suspension; A7) optionally isolating, washing and/or drying the nano-/microspheres.

In one embodiment, at room temperature, all the silica precursors are hydrolyzed independently with agitation at the stirring rate of at least 500 rpm for minimum 1 hour and combined into one container. (A0)

In one embodiment, all the pre-hydrolyzed silica precursors (A0) are combined into one container and used as said dispersed phase without any further treatment (e.g. solvent elimination, pre-condensation). (A1)

In one embodiment, the desired quantity of volatile solvents from the hydrolytic media can be removed by: i) evaporation under reduced pressure with rotary evaporator from room temperature to 70° C. or ii) distillation at the preferred temperature from 90 to 120° C., lower and higher temperature will be applied if it is needed. (A2)

In one embodiment, water miscible solvent is introduced in the dispersed phase, such as dimethyl sulfoxide (DMSO). (A3)

In one embodiment, the emulsification of the dispersed phase (A1 or A2 or A3) in the continuous phase can be realized with a rotor-stator homogenizer which generates stable microdroplets. In one embodiment, the emulsification of the dispersed phase (A1 or A2 or A3) in the continuous phase can be done with a sonic dismembrator which generates stable nanodroplets. (A4)

In one embodiment, during the emulsification the condensation catalyst is added to the emulsion and the emulsification process is maintained during 15 to 60 s to obtain the nano-/microspheres suspension. The condensation catalyst volume is added to reach a pH of the suspension at 9.0-11.5. (A5)

In one embodiment, after step (A5) optionally adding silica precursors with or without pre-hydrolyzation for delayed external surface functionalization.

In one embodiment, the nano-/microspheres suspension is aged at room temperature with stirring or shaking to maintain the stable suspension and avoid aggregation for 12 to 24 h. (A6)

In one embodiment, the nano-/microspheres are isolated by filtration for microspheres or isolated by centrifugation preferably from 5K to 100K G, and for example 15K G for 10 min for nanospheres. In one embodiment, nano-/microspheres are washed with organic solvents and water alternatively until the supernatant reaches neutrality (i.e. pH of about 7). Finally, the resulting material can be dried at room temperature or up to 70° C., at atmospheric pressure or under reduced pressure, for example for one day or more. (A7)

(Method B) In one embodiment, the process of preparation of silica nano-/microspheres with actives/payloads comprises, B0) separately hydrolyzing one or more silica precursor in a hydrolytic media to provide one or more pre-hydrolyzed silica precursor; B1) combining the pre-hydrolyzed silica precursors of step B0) to provide a dispersed phase comprising combined pre-hydrolyzed silica precursors; or B2) removing a part or totality of volatile solvents from said combined pre-hydrolyzed silica precursors to provide a dispersed phase comprising pre-condensed silica precursors; or B3) preparing a dispersed phase comprising a hydrophilic solvent by adding said hydrophilic solvent to said dispersed phase comprising combined pre-hydrolyzed silica precursors obtained in step B1) or by adding said hydrophilic solvent to said dispersed phase comprising pre-condensed silica precursors obtained in step B2); B4) emulsifying, in absence of a surfactant, the dispersed phase of the step B1), B2) or B3) in the continuous phase to provide a water in oil emulsion; B5) adding a condensation catalyst to the emulsion of step B4) to provide said organosiloxane nano-/microspheres; B6) optionally aging the suspension; B7) optionally isolating, washing and/or drying the nano-/microspheres. Dependently of the solubility of the actives/payloads, they can be introduced in the step (B1), (B2), (B3), (B4) or/and (B5) of the process.

In one embodiment, at room temperature, all the silica precursors are hydrolyzed independently with agitation at the stirring rate of at least 500 rpm for minimum 1 hour and combined into one container. The actives/payloads can be solubilized, dispersed or emulsified in the dispersed phase. (B0)

In one embodiment, all the pre-hydrolyzed silica precursors (B0) are combined into one container and used as said dispersed phase without any further treatment (e.g. solvent elimination, pre-condensation). (B1)

In one embodiment, the desired quantity of volatile solvents from the hydrolytic media can be removed by: i) evaporation under reduced pressure with rotary evaporator from room temperature to 70° C. or ii) distillation at the preferred temperature from 90 to 120° C., lower and higher temperature will be applied if it is needed. The actives/payloads can be solubilized, dispersed or emulsified in the resulting dispersed phase. (B2)

In one embodiment, water miscible solvent is introduced to the dispersed phase (B1 or B2), such as dimethyl sulfoxide. The actives/payloads can be solubilized, dispersed or emulsified in the resulting dispersed phase. (B3)

In one embodiment, the emulsification of the dispersed phase optionally containing actives/payloads (B1 or B2 or B3) in the continuous phase can be realized with a rotor-stator homogenizer which generates stable microdroplets. In one embodiment, the emulsification of the dispersed phase optionally containing actives/payloads (B1 or B2 or B3) in the continuous phase can be done with a sonic dismembrator which generates stable nanodroplets. In one embodiment, the actives/payloads can be dispersed in the continuous phase as the solid state. In another embodiment, the actives/payloads solubilized in the water miscible solvent, can be added in the emulsion. (B4)

In one embodiment, the actives/payloads can be solubilized in the condensation catalyst. During the emulsification, the condensation catalyst is added to the emulsion and the emulsification process is maintained during 15 to 60 s to obtain the nano-/microspheres suspension. The condensation catalyst volume is added to reach a pH of the suspension at 9.0-11.5. (B5)

In one embodiment, after step (B5) optionally adding silica precursors with or without pre-hydrolyzation for delayed external surface functionalization.

In one embodiment, the nano-/microspheres suspension is aged at room temperature with stirring or shaking to maintain the stable suspension and avoid aggregation for 12 to 24 h. (B6)

In one embodiment, the nano-/microspheres are isolated by filtration for microspheres or isolated by centrifugation preferably from 5K to 100K G, and for example 15K G for 10 min for nanospheres. The nano-/microspheres are washed with a solvent with the least solubility for the actives/payloads to avoid leaching. Finally, the resulting material is dried at room temperature or up to 70° C. depending on the properties of the actives/payloads, at atmospheric pressure or under reduced pressure, for example for one day or more. (B7)

The trapped actives/payloads quantity in the nano-/microspheres is determined by analytical methods, such as high-performance liquid chromatography (HPLC), elemental analysis (EA) or thermogravimetric analysis (TGA).

The sequestration yield is defined by the following formula (equation 1). The experimental active mass corresponds to the active quantified by analytical methods. The theoretical active mass corresponds to initial introduced quantity. The sequestration yield is comprised from 70 to 100%.

$$Sequestration yield = \frac{{m}_{A c t i v e ( Experimental )}}{{m}_{A c t i v e ( Theoritical )}} \times 100 \qquad ( Equation 1 )$$

The loading capacity is defined by the following formula (equation 2). The experimental active mass corresponds to the active quantified by analytical methods. The total mass corresponds to the mass of resulting nano-/microspheres, excluded water content. The loading capacity is actives/payloads-dependent. In one embodiment, the loading capacity is from 0.1 wt % to 80 wt %.

$$Loading capacity = \frac{{m}_{A c t i ve ( Experimental )}}{{m}_{t o t a l}} \times 100 \qquad ( Equation 2 )$$

In all the embodiments, the porous structures of the nano-/microspheres are non-organised. The nitrogen adsorption/desorption isotherms determine the surface area of the nano-/microspheres, which is typically up to 1000 m2.g⁻¹.

The outer surface hydrophobic/hydrophilic property of the nano-/microspheres is the result of the concoction of the silica precursors or the silica precursor's mixture.

In one embodiment, the hydrophobic/hydrophilic property of nano-/microspheres can be controlled by the composition of the silica precursors, such as the tetramethoxysilane (TMOS), tetraethoxysilane (TEOS), methyltriethoxysilane (C1-TES), butyltriethoxysilane (C4-TES), octyltriethoxysilane (C8-TES), the octadecyltriethoxysilane (C18-TES), Dimethyloctadecyl [3-(trimethoxysilyl)propyl] ammonium chloride (DOAPS) and 3-dimethylaminopropyltrimethoxysilane (DMAM).

In one embodiment, when only TEOS is used as silica precursor without active/payload loading, the contact angle of the corresponding nano-/microspheres is of 0°-40° which indicates the fully hydrophilic outer surface property. In another embodiments, when C4-TES and/or C8-TES and/or C18-TES are used, mixed with other silica precursors or not, the resulting nano-/microspheres shows the contact angle in a range of 80° to 150°, which confirms the tunable external surface property of these matrices from hydrophilic to hydrophobic.

To confirm the above observation, the outer surface composition of the nano-/microspheres, analyzed by X-ray photoelectron spectroscopy (XPS), is compared with the elemental composition of the entire nano-/microspheres to confirm the hydrophobic/hydrophilic balance of the outer surface.

In one embodiment, DOAPS, a positively charged silica precursor with C18 alky chain is used and mixed with other silica precursors. A positive zeta potential typically from +10 to +55 eV is observed, which puts in evidence that the positively charged ammonium functions are accessible, owing to the presence of hydrophobic C18 alky chain, on the external surface of the nano-/microspheres.

Preparation of Organosiloxane Nano-/Microspheres without an Active/Payload

In one embodiment, organosiloxane nano-/microspheres are synthetized following the described procedure in method A, for which: 1) pre-hydrolyzed and non pre-condensed C18-TES and TEOS silica precursors are used at a preferably molar ratio of 1%-75%/99%-25%, preferentially 5%/95%; 2) the continuous phase is preferably toluene; and 3) the condensation catalyst is preferably NH₄OH.

In one embodiment, organosiloxane nano-/microspheres are synthetized following the described procedure in method A, for which: 1) pre-hydrolyzed and non pre-condensed C18-TES and TEOS silica precursors are used at a preferably molar ratio of 1%-75%/99%-25%, preferentially 5%/95%; 2) the continuous phase is preferably toluene; and 3) the condensation catalyst is preferably TEA.

In one embodiment, organosiloxane nano-/microspheres are synthetized following the described procedure in method A, for which: 1) pre-hydrolyzed and non pre-condensed C18-TES and TEOS silica precursors are used at a preferably molar ratio of 1%-75%/99%-25%, preferentially 5%/95%; 2) the continuous phase is composed preferably by 50%-100% xylene and 50%-0% cyclohexane, preferentially 100% xylene; and 3) the condensation catalyst is preferably NH₄OH.

In one embodiment, organosiloxane nano-/microspheres are synthetized following the described procedure in method A, for which: 1) pre-hydrolyzed and non pre-condensed C18-TES and TEOS silica precursors are used at a preferably molar ratio of 1%-75%/99%-25%, preferentially 5%/95%; 2) the continuous phase is composed preferably by 50%-100% xylene and 50%-0% cyclohexane, preferentially 100% xylene; and 3) the condensation catalyst is preferably TEA.

In one embodiment, organosiloxane nano-/microspheres are synthetized following the described procedure in method A, for which: 1) pre-hydrolyzed and non pre-condensed C18-TES and TEOS silica precursors are used at a preferably molar ratio of 1%-75%/99%-25%, preferentially 5%/95%; 2) the continuous phase is preferably sunflower; and 3) the condensation catalyst is preferably NH₄OH.

In one embodiment, organosiloxane nano-/microspheres are synthesized following the described procedure in method A, for which: 1) the pre-hydrolyzed and pre-condensed C18-TES and TEOS silica precursors are used at a preferably molar ratio of 1%-75%/99%-25%; 2) the continuous phase is preferably cyclohexane; and 3) the condensation catalyst is preferably NH₄OH.

In one embodiment, organosiloxane nano-/microspheres are synthesized following the described procedure in method A, for which: 1) the pre-hydrolyzed and pre-condensed C18-TES and TEOS silica precursors are used at a preferably molar ratio of 1%-75%/99%-25%; 2) the continuous phase is preferably cyclohexane; and 3) the condensation catalyst is preferably TEA.

In one embodiment, organosiloxane nano-/microspheres are synthesized following the described procedure in method A, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is preferably toluene; and 3) the condensation catalyst is preferably NH₄OH.

In one embodiment, organosiloxane nano-/microspheres are synthesized following the described procedure in method A, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is preferably toluene; and 3) the condensation catalyst is preferably TEA.

In one embodiment, organosiloxane nano-/microspheres are synthesized following the described procedure in method A, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is composed preferably by 50%-100% xylene and 50%-0% cyclohexane, preferentially 100% xylene; and 3) the condensation catalyst is preferably NH₄OH.

In one embodiment, organosiloxane nano-/microspheres are synthesized following the described procedure in method A, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is composed preferably by 50%-100% xylene and 50%-0% cyclohexane, preferentially 100% xylene; and 3) the condensation catalyst is preferably TEA.

In one embodiment, organosiloxane nano-/microspheres are synthetized following the described procedure in method A, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is preferably toluene; and 3) the condensation catalyst is preferably NH₄OH.

In one embodiment, organosiloxane nano-/microspheres are synthetized following the described procedure in method A, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is preferably toluene; and 3) the condensation catalyst is preferably TEA.

In one embodiment, organosiloxane nano-/microspheres are synthetized following the described procedure in method A, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is composed preferably by 50%-100% xylene and 50%-0% cyclohexane, preferentially 100% xylene; and 3) the condensation catalyst is preferably NH₄OH.

In one embodiment, organosiloxane nano-/microspheres are synthetized following the described procedure in method A, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is composed preferably by 50%-100% xylene and 50%-0% cyclohexane, preferentially 100% xylene; and 3) the condensation catalyst is preferably TEA.

In one embodiment, organosiloxane nano-/microspheres are synthesized following the described procedure in method A, for which: 1) the pre-hydrolyzed and non pre-condensed C4-TES and TEOS silica precursors are used at a preferably molar ratio of 1%-75%/99%-25%, preferentially a molar ratio of 10%-50%/90%-50%; 2) the continuous phase is preferably toluene; and 3) the condensation catalyst is preferably NH₄OH.

In one embodiment, organosiloxane nano-/microspheres are synthesized following the described procedure in method A, for which: 1) the pre-hydrolyzed and non pre-condensed C4-TES and TEOS silica precursors are used at a preferably molar ratio of 1%-75%/99%-25%, preferentially a molar ratio of 10%-50%/90%-50%; 2) the continuous phase is preferably toluene; and 3) the condensation catalyst is preferably TEA.

In one embodiment, organosiloxane nano-/microspheres are synthesized following the described procedure in method A, for which: 1) the pre-hydrolyzed and non pre-condensed C4-TES and TEOS silica precursors are used at a preferably molar ratio of 1%-75%/99%-25%, preferentially a molar ratio of 10%-50%/90%-50%; 2) the continuous phase is composed preferably by 50%-100% xylene and 50%-0% cyclohexane, preferentially 100% xylene; and 3) the condensation catalyst is preferably NH₄OH.

In one embodiment, organosiloxane nano-/microspheres are synthesized following the described procedure in method A, for which: 1) the pre-hydrolyzed and non pre-condensed C4-TES and TEOS silica precursors are used at a preferably molar ratio of 1%-75%/99%-25%, preferentially a molar ration of 10%-50%/90%-50%; 2) the continuous phase is composed preferably by 50%-100% xylene and 50%-0% cyclohexane, preferentially 100% xylene; and 3) the condensation catalyst is preferably TEA.

In one embodiment, organosiloxane nano-/microspheres are synthesized following the described procedure in method A, for which: 1) DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is preferably toluene; and 3) the condensation catalyst is preferably NH₄OH. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is preferably toluene; and 3) the condensation catalyst is preferably TEA. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is composed preferably by 50%-100% xylene and 50%-0% cyclohexane, preferentially 100% xylene; and 3) the condensation catalyst is preferably NH₄OH. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is composed preferably by 50%-100% xylene and 50%-0% cyclohexane, preferentially 100% xylene; and 3) the condensation catalyst is preferably TEA. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C18-TES, DOAPS and TEOS silica precursors are used at a preferably molar ratio of 1%-75%/1%-75%/99%-25%, preferentially a molar ratio of 5%/5%/90%; 2) the continuous phase is preferably toluene; and 3) the condensation catalyst is preferably NH₄OH. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C18-TES, DOAPS and TEOS silica precursors are used at a preferably molar ratio of 0%-75%/0%-75%/99%-25%, preferentially a molar ratio of 5%/5%/90%; 2) the continuous phase is preferably toluene; and 3) the condensation catalyst is preferably TEA. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C18-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably NH₄OH. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C18-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably TEA. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C8-TES, DOAPS and TEOS silica precursors are used at a preferably molar ratio of 0%-75%/0%-75%/99%-25%, preferentially a molar ratio of 5%/5%/90%; 2) the continuous phase is preferably toluene; and 3) the condensation catalyst is preferably NH₄OH. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C8-TES, DOAPS and TEOS silica precursors are used at a preferably molar ratio of 0%-75%/0%-75%/99%-25% and a preferentially a molar ratio of 5%/5%/90%; 2) the continuous phase is preferably toluene; and 3) the condensation catalyst is preferably TEA. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C8-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably NH₄OH. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed C8-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably TEA. The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and pre-condensed C1-TES, C8-TES and TEOS silica precursors are used at a preferably molar ratio of 10%-20%/2.5%-7.5%/90%-60%, preferentially a molar ratio of 22.5%/7.5%/70%; 2) the continuous phase is preferably cyclohexane; and 3) the condensation catalyst is preferably NH₄OH.

In one embodiment, organosiloxane nano-/microspheres are synthesized following the described procedure in method A, for which: 1) the pre-hydrolyzed and pre-condensed C1-TES, C8-TES and TEOS silica precursors are used at a preferably molar ratio of 0%-75%/0%-75%/99%-25%, preferentially a molar ratio of 22.5%/7.5%/70%; 2) the continuous phase is preferably cyclohexane; and 3) the condensation catalyst is preferably TEA.

In one embodiment, organosiloxane nano-/microspheres are synthesized following the described procedure in method A, for which: 1) the pre-hydrolyzed and pre-condensed C1-TES, C8-TES and TEOS silica precursors are used at a preferably molar ratio of 10%-20%/2.5%-7.5%/90%-60%, preferentially a molar ratio of 22.5%/7.5%/70%; 2) the continuous phase is preferably cyclohexane; 3) the condensation catalyst is preferably NH₄OH; and 4) the DOAPS silica precursor is added in the suspension of nano-/microspheres at the weight ratio of 0.5-5%, preferably at the weight ratio of 2% (ratio of the weight of DOAPS to the weight of the pre-condensed silica precursors). The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and pre-condensed C1-TES, C8-TES and TEOS silica precursors are used at a preferably molar ratio of 0%-75%/0%-75%/99%-25% and a preferentially a molar ratio of 22.5%/7.5%/70%; 2) the continuous phase is preferably toluene; 3) the condensation catalyst is preferably TEA; and 4) the DOAPS silica precursor is added in the suspension of nano-/microspheres at the weight ratio of 0.5-5%, preferably at the weight ratio of 2% (ratio of the weight of DOAPS to the weight of the pre-condensed silica precursors). The resulting nano-/microspheres are characterized by a positive zeta potential, typically from +10 to +55 eV, once suspended in aqueous solution (water and phosphate buffered saline).

In one embodiment, organosiloxane nano-/microspheres are synthesized following the described procedure in method A, for which: 1) the pre-hydrolyzed and non pre-condensed SH-TES and TEOS silica precursors are used at a preferably molar ratio of 0%-100%/100%-0%; 2) the continuous phase is preferably toluene; and 3) the condensation catalyst is preferably NH₄OH.

In one embodiment, organosiloxane nano-/microspheres are synthesized following the described procedure in method A, for which: 1) the pre-hydrolyzed and non pre-condensed SH-TES and TEOS silica precursors are used at a preferably molar ratio of 0%-100%/100%-0%; 2) the continuous phase is preferably toluene; and 3) the condensation catalyst is preferably TEA.

In one embodiment, organosiloxane nano-/microspheres are synthesized following the described procedure in method A, for which: 1) the pre-hydrolyzed and non pre-condensed SH-TES and TEOS silica precursors are used at a preferably molar ratio of 0%-100%/100%-0%; 2) the continuous phase is composed preferably by 50%-100% xylene and 50%-0% cyclohexane, preferentially 100% xylene; and 3) the condensation catalyst is preferably NH₄OH.

In one embodiment, organosiloxane nano-/microspheres are synthesized following the described procedure in method A, for which: 1) the pre-hydrolyzed and non pre-condensed SH-TES and TEOS silica precursors are used at a preferably molar ratio of 0%-100%/100%-0%; 2) the continuous phase is composed preferably by 50%-100% xylene and 50%-0% cyclohexane, preferentially 100% xylene; and 3) the condensation catalyst is preferably TEA.

Preparation of Organosiloxane Nano-/Microspheres with an Active/Payload

In one embodiment, organosiloxane nano-/microspheres are synthetized following the described procedure in method B, for which: 1) pre-hydrolyzed and non pre-condensed C18-TES and TEOS silica precursors are used at a preferably molar ratio of 1%-75%/99%-25%, preferentially 5%/95%; 2) the continuous phase is preferably toluene; 3) the condensation catalyst is preferably NH₄OH; and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthetized following the described procedure in method B, for which: 1) pre-hydrolyzed and non pre-condensed C18-TES and TEOS silica precursors are used at a preferably molar ratio of 1%-75%/99%-25%, preferentially 5%/95%; 2) the continuous phase is preferably toluene; and 3) the condensation catalyst is preferably TEA; and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthetized following the described procedure in method B, for which: 1) pre-hydrolyzed and non pre-condensed C18-TES and TEOS silica precursors are used at a preferably molar ratio of 1%-75%/99%-25%, preferentially 5%/95%; 2) the continuous phase is composed preferably by 50%-100% xylene and 50%-0% cyclohexane, preferentially 100% xylene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthetized following the described procedure in method B, for which: 1) pre-hydrolyzed and non pre-condensed C18-TES and TEOS silica precursors are used at a preferably molar ratio of 1%-75%/99%-25%, preferentially 5%/95%; 2) the continuous phase is composed preferably by 50%-100% xylene and 50%-0% cyclohexane, preferentially 100% xylene; and 3) the condensation catalyst is preferably TEA, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthetized following the described procedure in method B, for which: 1) pre-hydrolyzed and non pre-condensed C18-TES and TEOS silica precursors are used at a preferably molar ratio of 1%-75%/99%-25%, preferentially 5%/95%; 2) the continuous phase is preferably sunflower; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and pre-condensed C18-TES and TEOS silica precursors are used at a preferably molar ratio of 1%-75%/99%-25%; 2) the continuous phase is preferably cyclohexane; and 3) the condensation catalyst is preferably NH₄OH, and 4) wherein this nano-/microspheres contain a hydrophilic active/payload. This hydrophilic active/payload can be introduced by: a) solubilisation or dispersion in the pre-condensed dispersed phase, the hydrophilic active/payload is preferentially 5-FU with a sequestration yield of 100% and a loading capacity up to 10%, preferentially at 5%; or b) solubilisation in the pre-condensed dispersed phase by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and pre-condensed C18-TES and TEOS silica precursors are used at a preferably molar ratio of 1%-75%/99%-25%; 2) the continuous phase is preferably cyclohexane; and 3) the condensation catalyst is preferably TEA, and 4) wherein this nano-/microspheres contain a hydrophilic active/payload. This hydrophilic active/payload can be introduced by: a) solubilisation or dispersion in the pre-condensed dispersed phase, the hydrophilic active/payload is preferentially 5-FU with a sequestration yield of 100% and a loading capacity up to 10%, preferentially at 5%; or b) solubilisation in the pre-condensed dispersed phase by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is preferably toluene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is preferably toluene; and 3) the condensation catalyst is preferably TEA and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is composed preferably by 50%-100% xylene and 50%-0% cyclohexane, preferentially 100% xylene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C8-TES and TEOS silica precursors are used at a preferably molar ratio of 1%-75%/99%-25%, preferentially a molar ratio of 10%/90%; 2) the continuous phase is composed preferably by 50%-100% xylene and 50%-0% cyclohexane, preferentially 100% xylene; and 3) the condensation catalyst is preferably TEA, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthetized following the described procedure in method B, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is preferably toluene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthetized following the described procedure in method B, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is preferably toluene; and 3) the condensation catalyst is preferably TEA and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthetized following the described procedure in method B, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is composed preferably by 50%-100% xylene and 50%-0% cyclohexane, preferentially 100% xylene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthetized following the described procedure in method B, for which: 1) pre-hydrolyzed and non pre-condensed C8-TES, C18-TES and TEOS silica precursors are used at a molar ratio of 1%-75%/1%-75%/99%-25%, preferably 5%/5%/90%; 2) the continuous phase is composed preferably by 50%-100% xylene and 50%-0% cyclohexane, preferentially 100% xylene; and 3) the condensation catalyst is preferably TEA, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthesized following the described procedure in method B, for which: 1) the pre-hydrolyzed and non pre-condensed C4-TES and TEOS silica precursors are used at a preferably molar ratio of 1%-75%/99%-25%, preferentially a molar ratio of 10%-50%/90%-50%; 2) the continuous phase is preferably toluene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C4-TES and TEOS silica precursors are used at a preferably molar ratio of 1%-75%/99%-25%, preferentially a molar ratio of 10%-50%/90%-50%; 2) the continuous phase is preferably toluene; and 3) the condensation catalyst is preferably TEA, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and non pre-condensed C4-TES and TEOS silica precursors are used at a preferably molar ratio of 1%-75%/99%-25%, preferentially a molar ratio of 10%-50%/90%-50%; 2) the continuous phase is composed preferably by 50%-100% xylene and 50%-0% cyclohexane, preferentially 100% xylene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C4-TES and TEOS silica precursors are used at a preferably molar ratio of 1%-75%/99%-25%, preferentially a molar ration of 10%-50%/90%-50%; 2) the continuous phase is composed preferably by 50%-100% xylene and 50%-0% cyclohexane, preferentially 100% xylene; and 3) the condensation catalyst is preferably TEA, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthesized following the described procedure in method B, for which: 1) DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is preferably toluene; and 3) the condensation catalyst is preferably NH₄OH. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is preferably toluene; and 3) the condensation catalyst is preferably TEA. The resulting nano-/microspheres are characterized by a positive zeta potential, typically from +10 to +55 eV, once suspended in aqueous solution (water and phosphate buffered saline); and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is composed preferably by 50%-100% xylene and 50%-0% cyclohexane, preferentially 100% xylene; and 3) the condensation catalyst is preferably NH₄OH. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: DOAPS (the pre-hydrolyzed or not) and pre-hydrolyzed TEOS silica precursors are used non pre-condensed at a preferably molar ratio of 1%-75%/99%-25%, preferentially a molar ratio of 1%-20%/99%-80%; 2) the continuous phase is composed preferably by 50%-100% xylene and 50%-0% cyclohexane, preferentially 100% xylene; and 3) the condensation catalyst is preferably TEA. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthesized following the described procedure in method B, for which: 1) the pre-hydrolyzed and non pre-condensed C18-TES, DOAPS and TEOS silica precursors are used at a preferably molar ratio of 1%-75%/1%-75%/99%-25%, preferentially a molar ratio of 5%/5%/90%; 2) the continuous phase is preferably toluene; and 3) the condensation catalyst is preferably NH₄OH. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C18-TES, DOAPS and TEOS silica precursors are used at a preferably molar ratio of 0%-75%/0%-75%/99%-25%, preferentially a molar ratio of 5%/5%/90%; 2) the continuous phase is preferably toluene; and 3) the condensation catalyst is preferably TEA. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and non pre-condensed C18-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably NH₄OH. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C18-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably TEA. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthesized following the described procedure in method B, for which: 1) the pre-hydrolyzed and non pre-condensed C8-TES, DOAPS and TEOS silica precursors are used at a preferably molar ratio of 0%-75%/0%-75%/99%-25%, preferentially a molar ratio of 5%/5%/90%; 2) the continuous phase is preferably toluene; and 3) the condensation catalyst is preferably NH₄OH. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C8-TES, DOAPS and TEOS silica precursors are used at a preferably molar ratio of 0%-75%/0%-75%/99%-25% and a preferentially a molar ratio of 5%/5%/90%; 2) the continuous phase is preferably toluene; and 3) the condensation catalyst is preferably TEA. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20.

In one embodiment, organosiloxane nano-/microspheres are synthesized following the described procedure in method B, for which: 1) the pre-hydrolyzed and non pre-condensed C8-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably NH₄OH. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed C8-TES, DOAPS and TEOS silica precursors are used at a preferably molar ratio of 0%-75%/0%-75%/99%-25%, preferentially a molar ratio of 5%/5%/90%; 2) the continuous phase is composed preferably by 50%-100% xylene and 50%-0% cyclohexane, preferentially 100% xylene; and 3) the condensation catalyst is preferably TEA. The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

In one embodiment, organosiloxane nano-/microspheres are synthesized following the described procedure in method B, for which: 1) the pre-hydrolyzed and pre-condensed C1-TES, C8-TES and TEOS silica precursors are used at a preferably molar ratio of 10%-20%/2.5%-7.5%/90%-60%, preferentially a molar ratio of 22.5%/7.5%/70%; 2) the continuous phase is preferably cyclohexane; and 3) the condensation catalyst is preferably NH₄OH, and 4) wherein this nano-/microspheres contain a hydrophilic active/payload. This hydrophilic active/payload can be introduced by: a) solubilisation or dispersion in the pre-condensed dispersed phase, the hydrophilic active/payload is preferentially 5-FU with a sequestration yield of 100% and a loading capacity up to 10%, preferentially at 5%; or b) solubilisation in the pre-condensed dispersed phase by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and pre-condensed C1-TES, C8-TES and TEOS silica precursors are used at a preferably molar ratio of 0%-75%/0%-75%/99%-25%, preferentially a molar ratio of 22.5%/7.5%/70%; 2) the continuous phase is preferably cyclohexane; and 3) the condensation catalyst is preferably TEA, and 4) wherein this nano-/microspheres contain a hydrophilic active/payload. This hydrophilic active/payload can be introduced by: a) solubilisation or dispersion in the pre-condensed dispersed phase, the hydrophilic active/payload is preferentially 5-FU with a sequestration yield of 100% and a loading capacity up to 10%, preferentially at 5%; or b) solubilisation in the pre-condensed dispersed phase by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and pre-condensed C1-TES, C8-TES and TEOS silica precursors are used at a preferably molar ratio of 10%-20%/2.5%-7.5%/90%-60%, preferentially a molar ratio of 22.5%/7.5%/70%; 2) the continuous phase is preferably cyclohexane; 3) the condensation catalyst is preferably NH₄OH; and 4) the DOAPS silica precursor is added in the suspension of nano-/microspheres at the weight ratio of 0.5-5%, preferably at the weight ratio of 2% (ratio of the weight of DOAPS to the weight of the pre-condensed silica precursors). The resulting nano-/microspheres are characterized by a positive zeta potential, typically from +10 to +55 eV, once suspended in aqueous solution (water and phosphate buffered saline); and 4) wherein this nano-/microspheres contain a hydrophilic active/payload. This hydrophilic active/payload can be introduced by: a) solubilisation or dispersion in the pre-condensed dispersed phase, the hydrophilic active/payload is preferentially 5-FU with a sequestration yield of 100% and a loading capacity up to 10%, preferentially at 5%; or b) solubilisation in the pre-condensed dispersed phase by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and pre-condensed C1-TES, C8-TES and TEOS silica precursors are used at a preferably molar ratio of 0%-75%/0%-75%/99%-25% and a preferentially a molar ratio of 22.5%/7.5%/70%; 2) the continuous phase is preferably toluene; 3) the condensation catalyst is preferably TEA; and 4) the DOAPS silica precursor is added in the suspension of nano-/microspheres at the weight ratio of 0.5-5%, preferably at the weight ratio of 2% (ratio of the weight of DOAPS to the weight of the pre-condensed silica precursors). The resulting nano-/microspheres are characterized by a positive zeta potential, typically from +10 to +55 eV, once suspended in aqueous solution (water and phosphate buffered saline), and 4) wherein this nano-/microspheres contain a hydrophilic active/payload. This hydrophilic active/payload can be introduced by: a) solubilisation or dispersion in the pre-condensed dispersed phase, the hydrophilic active/payload is preferentially 5-FU with a sequestration yield of 100% and a loading capacity up to 10%, preferentially at 5%; or b) solubilisation in the pre-condensed dispersed phase by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and non pre-condensed SH-TES and TEOS silica precursors are used at a preferably molar ratio of 0%-100%/100%-0%; 2) the continuous phase is preferably toluene; and 3) the condensation catalyst is preferably NH₄OH, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%; or c) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed SH-TES and TEOS silica precursors are used at a preferably molar ratio of 0%-100%/100%-0%; 2) the continuous phase is preferably toluene; and 3) the condensation catalyst is preferably TEA, and 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) dispersion in the continuous phase before emulsification process. The hydrophilic active/payload is preferentially 5-FU with a sequestration yield of 100% and a loading capacity up to 50%, preferentially at 20%.

In one embodiment, organosiloxane nano-/microspheres are synthesized following the described procedure in method B, for which: 1) the pre-hydrolyzed and non pre-condensed SH-TES and TEOS silica precursors are used at a preferably molar ratio of 0%-100%/100%-0%; 2) the continuous phase is composed preferably by 50%-100% xylene and 50%-0% cyclohexane, preferentially 100% xylene; and 3) the condensation catalyst is preferably NH₄OH, 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%; or e) solubilisation in the condensation catalyst. The hydrophilic active/payload is preferentially 5-FU with a sequestration yield of 70-90% and a loading capacity up to 10%, preferentially at 5%.

In one embodiment, organosiloxane nano-/microspheres are synthesized following the described procedure in method B, for which: 1) the pre-hydrolyzed and non pre-condensed SH-TES and TEOS silica precursors are used at a preferably molar ratio of 0%-100%/100%-0%; 2) the continuous phase is composed preferably by 50%-100% xylene and 50%-0% cyclohexane, preferentially 100% xylene; and 3) the condensation catalyst is preferably TEA, 4) wherein this nano-/microspheres contain a hydrophilic active/payload. This hydrophilic active/payload can be introduced by: a) solubilisation or dispersion in the pre-hydrolyzed silica precursors, the hydrophilic active/payload is preferentially 5-FU with a sequestration yield of 100% and a loading capacity up to 10%, preferentially at 5%; or b) solubilisation in the pre-hydrolyzed silica precursors by the help of a hydrophilic co-solvent and preferentially the hydrophilic co-solvent is DMSO. The hydrophilic active/payload is preferentially 5-FU with a sequestration yield of 100% and a loading capacity up to 50%, preferentially at 20%; or c) dispersion in the continuous phase before emulsification process. The hydrophilic active/payload is preferentially 5-FU with a sequestration yield of 100% and a loading capacity up to 50%, preferentially at 20%; or d) solubilisation in the hydrophilic co-solvent preferentially DMSO and addition to the emulsion. The hydrophilic active/payload is preferentially 5-FU with a sequestration yield of 100% and a loading capacity up to 10%, preferentially at 5%.

Examples of Nano-/Microspheres Obtained with Method a (I.E., without the Presence of Active/Payload):

EXAMPLE 1: Preparation of microspheres with fully hydrolyzed and non pre-condensed C18-TES and TEOS (Method A), in toluene.

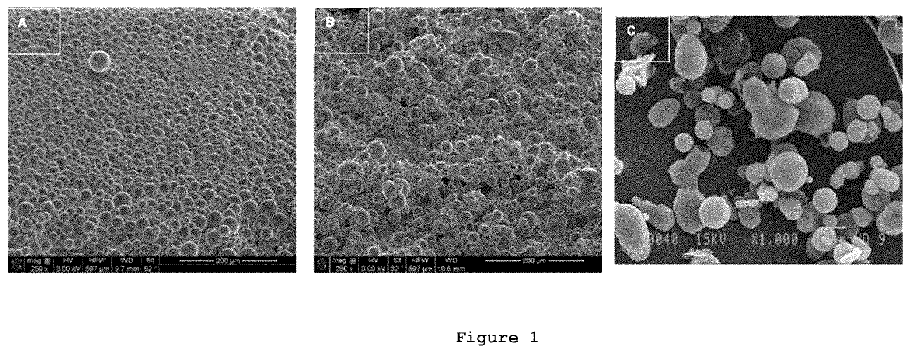

EXAMPLE 1-1: Microspheres with C18-TES/TEOS molar ratio=1%/99%. Typically, 11.02 g (57.7 mmol) of TEOS was pre-hydrolyzed in acidic conditions of 6.18 g of 0.01 N HCl for 1 hour under stirring. Meanwhile, the pre-hydrolyzation of 2.45 g of C18-TES was carried out in the mixture of 0.69 g of 0.05 N HCl, 4.99 g of THF and/or without 0.5 mL of EtOH in another vial under stirring for 1 hour. The pre-hydrolyzed C18-TES silica precursor was then added to the pre-hydrolyzed TEOS and stirred at room temperature for 15-30 min, leading to the formation of hydrolyzed 1%/99% C18-TES/TEOS silica precursors. This obtained dispersed phase was added to 200 g of toluene, as continuous phase, under mixing with Ultra-Turrax homogenizer (Ultra-Turrax® T 25 coupled with S25N-18G) at high speed of (7K-15K) rpm. The mixture was stirred for 5 min to generate a homogeneous emulsion. Then, 1 g of concentrated NH₄OH was added as condensation catalyst. After 1 min, the Ultra-Turrax was stopped and the suspension was kept under gentle agitation for at least overnight. After that, the suspension of microspheres was filtered, washed with toluene and ethanol, and finally dried at room temperature for 3 days. The morphological and textural characteristics of the obtained spheres are shown in FIG. 1 and Table 1.

EXAMPLE 1-2: Microspheres with C18-TES/TEOS molar ratio=10%/90%.

Silica microspheres containing x % C18-TES and y % TEOS were prepared by using the same procedure as described in Example 1-1. The main characteristics of the obtained spheres are summarised in FIG. 1 and Table 1.

EXAMPLE 1-3: Microspheres with C18-TES/TEOS molar ratio=75%/25%.

Silica microspheres containing 75% C18-TES and 25% TEOS were prepared by using the same procedure as described in Example 1-1. The main characteristics of the obtained microspheres are reported in FIG. 1 and Table 1.

TABLE 1
size and porosity data of examples of microspheres:

| | | | Porosity | | |
|---|---|---|---|---|---|
| | Particle size | | | | Average |
| | d50 | d90/ | Surface area | Pore volume | pore size |
| Example | (μm) | d10 | (m² · g⁻¹) | (cm³ · g⁻¹) | (nm) |
| Example 1-1 | 23 | 8 | 358 | 0.53 | 6.1 |
| Example 1-2 | 19 | 8 | 405 | 0.73 | 7.2 |
| Example 1-3 | 12 | 4 | 80 | 0.44 | 21.8 |

EXAMPLE 2: Preparation of microspheres with fully hydrolyzed and non pre-condensed C18-TES and TEOS (molar ratio: 7%/93%, method A), in other continuous phases.

EXAMPLE 2-1: In 75% Xylene/25% Cyclohexane.

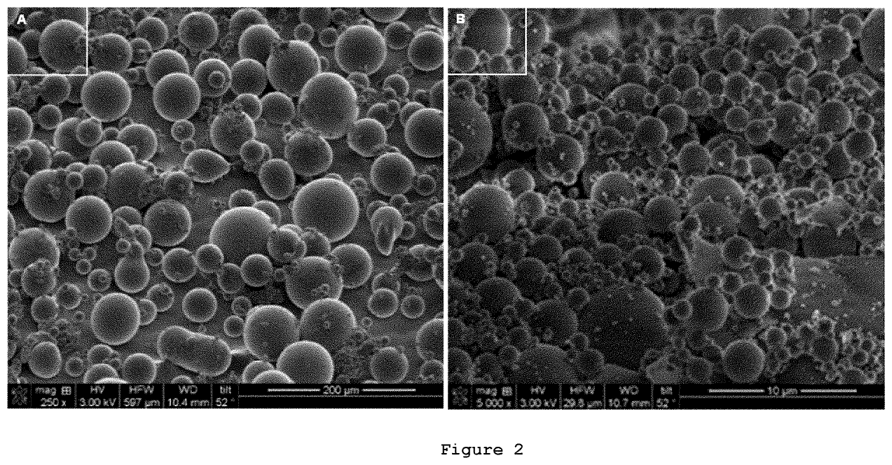

Silica microspheres containing 7% C18-TES and 93% TEOS were prepared by using the same procedure as described in Example 1.1 except that the mixture of hydrolyzed silanes was emulsified here in a mixture of xylene and cyclohexane (75% and 25%, respectively). The main microspheres characteristics are summarized in FIG. 2 and Table 2.

Example 2-2: In sunflower oil.

Silica microspheres containing 7% C18-TES and 93% TEOS were prepared by using the same procedure as described in Example 1.1 with the exception that the mixture of hydrolyzed silanes was emulsified here in sunflower oil. The main microspheres characteristics are summarized in FIG. 2 and Table 2.

TABLE 2
Morphological and textural characteristics of microspheres:

| | | | Porosity | | |
|---|---|---|---|---|---|
| | Particle size | | | | Average |
| | d50 | d90/ | Surface area | Pore volume | pore size |
| Example | (μm) | d10 | (m² · g⁻¹) | (cm³ · g⁻¹) | (nm) |
| Example 2-1 | 45 | 15 | 81 | 0.26 | 13 |
| Example 2-2 | 12 | 6 | 16 | 0.07 | 17 |

EXAMPLE 3: Preparation of microspheres with fully hydrolyzed and pre-condensed C18-TES and TEOS (molar ratio: 72%/28%, method A), in hexane.

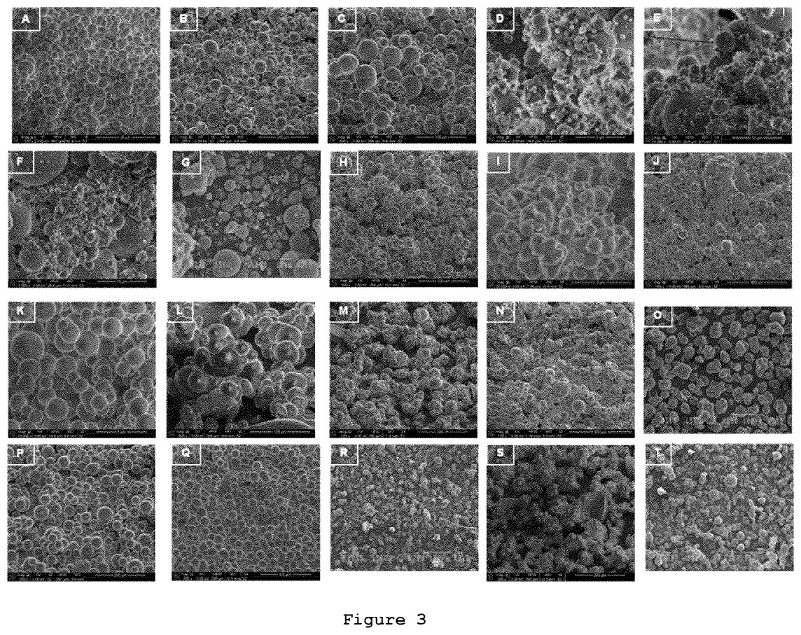

Typically, a 250 mL round bottle flask was first charged with 0.12 g of 0.01 N hydrochloric acid and 0.55 g of ethanol, followed by adding 1.23 g (5.9 mmol) of TEOS. In a 30 ml vial, 0.96 g (2.3 mmol) of C18-TES was combined with respectively 0.14 g of 0.05 N HCl, as well as 1.3 g of THF. These two mixtures were stirred for about 1.5 hour, and subsequently combined to 250 mL round bottle flask. The ethanol, which was both added and produced during the hydrolysis process, was gradually evaporated under reduced pressure at 40° C. to produce the dispersed phase, with viscosity of about 25 cp. After that, 3 g (38.3 mmol) of DMSO was added. To produce the water in oil (W/O) emulsion, 150 mL hexane, in a separate container, was stirred with Ultra-Turrax homogenizer at 18K rpm and the dispersed phase was then added. After continuous stirring for 5 min at 18K rpm, 1.6 ml (11.2 mmol) of NH₄OH (7 N in methanol) was introduced in the emulsion dropwise as condensation catalyst while stirring. The mixing was continued for 1 min. The resulting suspension was further aged at room temperature in a shaker at the speed of 200 rpm overnight. The product was filtered off and dried at room temperature for 3 days. The average particles size of the obtained spheres is d50=9 μm; d90/d10=9 (FIG. 3-A). EXAMPLE 4: Preparation of microspheres with fully hydrolyzed and non pre-condensed C8-TES and TEOS (molar ratio: 10%/90%, method A), in toluene.

Silica microspheres containing 10% C8-TES and 90% TEOS have been prepared by using the same procedure as described in Example 1-1. microspheres were obtained with an average diameter of 23 μm (d50); d90/d10=9 (FIG. 3-B).

EXAMPLE 5: Preparation of microspheres with fully hydrolyzed and non pre-condensed C4-TES and TEOS (molar ratio: 10%/90%, method A), in toluene.

Silica microspheres containing 10% C4-TES and 90% TEOS were prepared by using the same procedure as described in Example 1-1. The average diameter of the resulted microspheres is d50=70 μm with d90/d10=8 (FIG. 3-C).

EXAMPLE 6: Preparation of microspheres with DOAPS and fully hydrolyzed and non pre-condensed TEOS (molar ratio: 10%/90%, method A), in toluene.

EXAMPLE 6.1: Using fully hydrolyzed and non pre-condensed DOAPS. Silica microspheres containing 10% DOAPS and 90% TEOS were prepared by using the same procedure as described in Example 1-1. Microspheres were obtained with an average particle size of 18 μm (d50), including the presence of a proportion of 200 nm nanospheres; d90/d10=18 (FIG. 3-D). Once suspended in water (pH=6), positively charged microspheres were generated with a zeta potential value of +55 mV. This demonstrates the presence of DOAPS molecules at the external outer surface of the microspheres.

EXAMPLE 6.2: Using non-hydrolyzed and non pre-condensed DOAPS. Silica microspheres containing 10% DOAPS and 90% TEOS were prepared by using the same procedure as described in Example 1-1. Microspheres were obtained with an average particle size of 15 μm (d50), including the presence of a proportion of 200 nm nanospheres; d90/d10=18 (FIG. 3-E). Once suspended in water (pH=6), negatively charged microspheres were generated with a zeta potential value of −20 mV. This suggests that the passively charge of DOAPS is not accessible at the external outer surface of the microspheres, taking into account the CNS result which confirms the presence of DOAPS in the resulted spheres (% C_{(obtained by CNS)}=28.5% versus % C_{(theoretically)}=29% and (% N_{(obtained by CNS)}=1.3% versus % C_{(theoretically)}=1.5%) and the hydrophobic contact angle value (131°).

EXAMPLE 7: Preparation of microspheres with fully hydrolyzed and non pre-condensed DMAM and TEOS (molar ratio: 10%/90%, method A), in toluene.

Silica microspheres containing 10% DMAM and 90% TEOS were prepared by using the same procedure as described in Example 1-1. Microspheres were obtained with an average particle size of 67 μm (d50); d90/d10=45 (FIG. 3-F).

EXAMPLE 8: Preparation of microspheres with fully hydrolyzed and non pre-condensed SH-TES (mercaptopropyltriethoxysilane) and TEOS (molar ratio: 19%/81%, method A), in toluene.

Silica microspheres containing 19% SH-TES and 81% TEOS have been prepared by using the same procedure as described in Example 1-1. The average diameter of the obtained microspheres is d50=34 μm with d90/d10=29(FIG. 3-G).

EXAMPLE 9: Preparation of microspheres with fully hydrolyzed and non pre-condensed 7-Bromoheptyltrimethoxysilane (BrC₇-TES) and TEOS (molar ratio: 50%/50%, method A), in toluene.

Silica microspheres containing 50% BrC₇-TES and 50% TEOS were prepared by using the same procedure as described in Example 1-1. Microspheres were obtained with an average particle size of 11 μm (d50); d90/d10=5 (FIG. 3-H).

EXAMPLE 10: Preparation of microspheres with fully hydrolyzed and pre-condensed: Triethoxy (trifluoromethyl) silane (CF₃-TES) and TEOS (molar ratio: 60%/40%, method A), in hexane.

Silica microspheres containing 60% CF₃-TES and 40% TEOS were prepared by using the same procedure as described in Example 3. This leads to the formation of microspheres with an average size of 40 μm (d50); d90/d10=4 (FIG. 3-I).

EXAMPLE 11: Preparation of microspheres with fully hydrolyzed and non pre-condensed diPh-DES (2-(Diphenylphosphino)ethyltriethoxysilane) and TEOS (molar ratio: 50%/50%, method A), in toluene.

Silica microspheres containing 50% diPh-DES and 50% TEOS were prepared by using the same procedure as described in Example 1.1. This leads to the obtention of microspheres with an average diameter of 68 μm (d50); d90/d10=2 (FIG. 3-J).

EXAMPLE 12: Preparation of microspheres with fully hydrolyzed and non pre-condensed C1-TES (100%, method A), in toluene.

Silica microspheres containing 100% C1-TES were prepared by using the same procedure as described in Example 1-1. The average diameter of the obtained microspheres is d50=3.5 μm with d90/d10=11 (FIG. 3-K).

EXAMPLE 13. Preparation of microspheres with fully hydrolyzed and non pre-condensed 100% BTES-ethane (100%, method A), in toluene.

Silica microspheres containing 100% BTES-ethane were prepared by using the same procedure as described in Example 1-1. Microspheres were obtained with an average diameter of 45 μm (d50); d90/10=39 (FIG. 3-L).

EXAMPLE 14. Preparation of microspheres with fully hydrolyzed and pre-condensed 100% BTES-ethylene (100%, method A), in hexane.

Silica microspheres containing 100% BTES-ethylene were prepared by using the same procedure as described in Example 3. Microspheres were obtained with an average size of 37 μm (d50); d90/d10=9 (FIG. 3-M).

EXAMPLE 15: Preparation of microspheres with fully hydrolyzed and non pre-condensed TEOS (100%, method A), in toluene.

Silica microspheres containing 100% TEOS were prepared by using the same procedure as described in Example 1-1. Microspheres were obtained with an average diameter of 104 μm (d50); d90/d10=111 (FIG. 3-N).

EXAMPLE 16: Preparation of microspheres with fully hydrolyzed and pre-condensed TEOS (100%, method A), in hexane.

Silica microspheres containing 100% TEOS were prepared by using the same procedure as described in Example 3. The resulted microspheres have an average particles size of 37 μm (d50); d90/d10=3 (FIG. 1-O).

EXAMPLE 17: Preparation of microspheres with the combination of several ormosils (≥2)

Example 17-1: Using fully hydrolyzed and non pre-condensed C8-TES, C18-TES and TEOS (molar ratio 5%/5%/90%, method A), in toluene.

Silica microspheres containing 5% C8-TES, 5% C18-TES and 90% TEOS were prepared by using the same procedure as described in Example 1.1. The average particles size of the obtained microspheres is d50=22 μm; d90/d10=6 (FIG. 3-P).

Example 17-2: Using fully hydrolyzed and pre-condensed TMS (Trimethylsilane), C8-TES and TEOS (molar ratio 22.5%/7.5%/70%, method A), in hexane.

Silica microspheres containing 22.5% TMS, 7.5% C8-TES and 70% TEOS were prepared by using the same procedure as described in Example 3. The average particles size of the obtained microspheres is d50=18 μm; d90/d10=2 (FIG. 3-Q).

Example 17-3: Using fully hydrolyzed and pre-condensed C1-TES, DOAPS and TEOS (molar ratio 22.5%/7.5%/70%, method A), in hexane.

Silica microspheres containing 22.5% C1-TES, 7.5% DOAPS and 70% TEOS were prepared by using the same procedure as described in Example 3. The average particles size of the resulted microspheres is d50=19 μm; d90/d10=3 (FIG. 3-R).

Example 17-4: Using fully hydrolyzed and pre-condensed BTES-ethylene, C1-TES and C8-TES (molar ratio 70%/22.5%/7.5%, method A), in hexane.

Silica microspheres containing 22.5% C1-TES, 7.5% DOAPS and 70% TEOS were prepared by using the same procedure as described in Example 3. The average particles size of the resulted microspheres is d50=19 μm; d90/d10=3 (FIG. 3-S).

EXAMPLE 18. Preparation of microspheres using primene as organic base, with fully hydrolyzed and pre-condensed C1-TES, C8-TES and TEOS (molar ratio 22.5%/7.5%/70%, method A), in hexane.

Silica microspheres containing 22.5% C1-TES, 7.5% C8-TES and 70% TEOS were prepared by using the same procedure as described in Example 3, with the exception that primene was used instead of NH₄OH as the condensation catalyst. The average particles size of the obtained microspheres is d50=18 μm; d90/d10=2 (FIG. 3-T).

EXAMPLE 19. Examples of physicochemical characterization of the microspheres.

The thermogravimetry analysis (TGA) shows the thermic degradation of organic groups between 180 and 500° C., which confirms the presence of organic molecules corresponding to the used organosilanes.

As XPS detects the presence of elements in a maximum depth of a few nanometers only, the analysis of Si(2s), C(1s), O(1s) and N(1s) peaks confirm the presence of the functional groups of the organosilanes on the outer surface of nano-/microspheres. These data (Table 3) reveal that the longer the alkyl chain is, the higher the carbon atomic percentage (% C) and the carbon-to-silicon ratio (C/Si) are. Interestingly, when DOAPS is used, XPS shows the apparition of quantifiable content of nitrogen element (Table 3, example 6-1). In addition, High-resolution analysis of the C peak confirmed only the presence of C—C and C—H binding (band at 285 eV) which is related to the C8 alkyl functional group for microspheres presented in example 4 (avec 10% C8-TES).

Furthermore, by comparing C/Si ratio obtained by XPS (analysis of the external surface, 5 nm in depth) with that of obtained by elemental analysis (CNS and XRF, analysis of entire nano-/microspheres), the significantly lower C/Si ratio found by elemental analysis confirms that the alkyl chains of the used organosilanes are principally on the outer surface of the nano-/microspheres.

TABLE 3
Elemental analysis

| | | | | | CNS | XRF | | |
|---|---|---|---|---|---|---|---|---|
| | XPS analysis* (5 nm depth) | | | | analysis | analysis | C/Si | C/Si |
| Example | % C | % N | % Si | % O | % C | % Si | (XPS) | (entire) |
| Example 5 | 20.4 | — | 21.2 | 58.4 | 6.3 | 35.2 | 0.96 | 0.17 |
| with C4-TES and | | | | | | | | |
| TEOS (10%/90%) | | | | | | | | |
| Example 4 | 34.9 | — | 17.2 | 47.6 | 13.3 | 28.8 | 2.03 | 0.46 |
| with C8-TES and | | | | | | | | |
| TEOS (10%/90%) | | | | | | | | |
| Example 6-1 | 47.2 | 1.6 | 12.3 | 38.5 | 26.3 | 26.6 | 3.31 | 0.99 |
| with DOAPS and | | | | | | | | |
| TEOS (10%/90%) | | | | | | | | |

*Traces of other elements could be present.

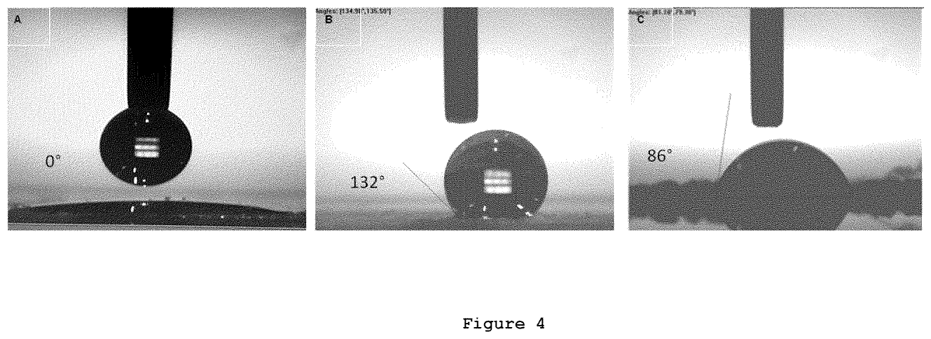

The measured contact angle confirms the tunable hydrophobic/hydrophilic property of the outer surface of the microspheres. Indeed, 1) fully hydrophilic external surface of microspheres was obtained with 100% TEOS (Example 15) having a contact angle less than 40° (FIG. 4-A), 2) fully hydrophobic external surface was obtained with C₈-TES (Example 4), having a contact angle 120-150° (FIG. 4-B), and 3) balanced hydrophilic/hydrophobic external surface with C₄-TES (Example 5), having a contact angle 80-90° (FIG. 4-C).

Examples of Nano-/Microspheres Obtained with Method B (I.E., with the Presence of Active/Payload):

EXAMPLE 20: Preparation of active/payload containing microspheres by the procedure of adding the active/payload in the dispersed phase (B1); active/payload trapped at solubilized state.

EXAMPLE 20-1: Preparation of D-Glucose containing microspheres with fully hydrolyzed and non pre-condensed C18-TES and TEOS (molar ratio: 5%/95%, method B).

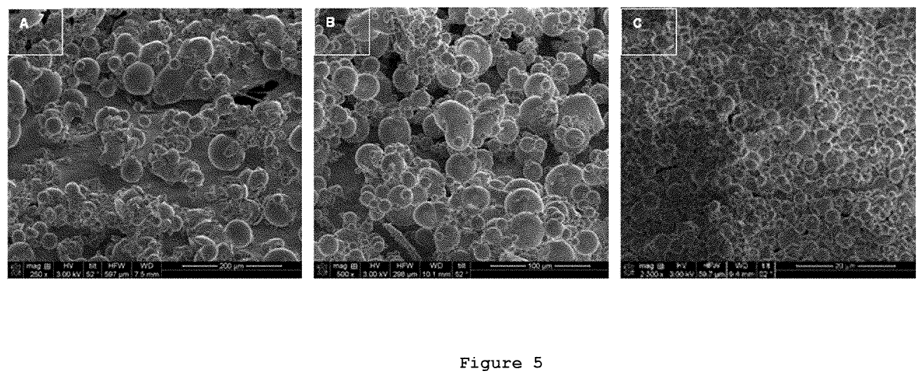

Microspheres with loading capacity of 33 wt % (Table 4) D-Glucose were prepared using the procedure described in Example 1.1, except that D-Glucose was solubilized into the dispersed phase (B1). The resulted microspheres have an average particle size of 50 μm (FIG. 5-A). After the extraction of the active (i.e. D-Glucose), the porosity data of the obtained microspheres are summarized in Table 4.

EXAMPLE 20-2: Preparation of uracil containing microspheres with fully hydrolyzed and non pre-condensed C18-TES, C8-TES and TEOS (molar ratio: 5%/5%/90%, method B).

Microspheres with loading capacity of 5 wt % (Table 4) uracil were prepared using the procedure described in Example 1.1, except that uracil was solubilized in DMSO as water miscible solvent and then added to the dispersed phase (B3), before emulsification step where xylene is used here as continuous phase. The obtained microspheres have an average particle size of 28 μm (FIG. 5-B). After the extraction of the active (i.e. uracil), the porosity data of the obtained microspheres are summarized in Table 4.

EXAMPLE 20-3: Preparation of uracil containing microspheres with fully hydrolyzed and pre-condensed C1-TES, C8-TES and TEOS (molar ratio: 22.5%/7.5%/70%, method B).

Microspheres with loading capacity of 9 wt % (Table 4) uracil were prepared using the procedure described in Example 3, except that uracil was solubilized dispersed phase after pre-condensation and before emulsification steps (B2); cyclohexane was used here as continuous phase. The obtained microspheres have an average particle size of 9 μm (d50) (FIG. 5-C). After the extraction of the active (i.e. uracil), the porosity data of the resulted microspheres are summarized in Table 4.

TABLE 4
Morphological and textural characteristics of the resulted microspheres:

| | Active loading | | | Porosity | | |
|---|---|---|---|---|---|---|
| | performance | | Particle | Surface | Pore | Average |
| | Loading | Sequestration | size | area | volume | pore size |
| Example | (wt %) | yield % | (d50, μm) | (m² · g⁻¹) | (cm³ · g⁻¹) | (nm) |
| Example 20-1 | 33 | 100 | 53 | 420 | 0.80 | 7.7 |
| Example 20-2 | 5 | 99 | 28 | 744 | 0.61 | 3.3 |
| Example 20-3 | 9 | 99 | 9 | 413 | 0.36 | 3.4 |

EXAMPLE 21: Preparation of active/payload containing microspheres by the procedure of adding the active/payload in dispersed phase; active/payload trapped at solid state.

EXAMPLE 21-1: Preparation of 5-FU containing microspheres with fully hydrolyzed and non pre-condensed C18-TES, C8-TES and TEOS (molar ratio: 5%/5%/90%, method B).

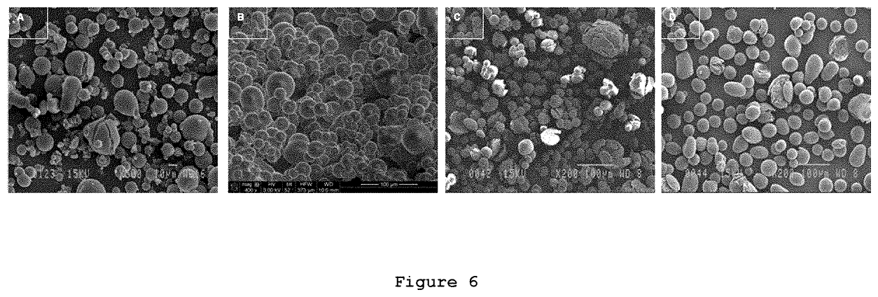

Microspheres with loading capacity of 20 wt % (Table 5) uracil are prepared using the procedure described in Example 1.1, except that 5-FU powder was suspended in the dispersed phase (B1), followed by adding DMSO before emulsification step (B3). Xylene was used here as continuous phase. The obtained spheres have an average particle size of 21 μm (FIG. 6-A). After the extraction of the active (i.e. uracil), the porosity data of the obtained microspheres are summarized in Table 5.

EXAMPLE 21-2: Preparation of uracil containing microspheres with fully hydrolyzed and pre-condensed C1-TES, C8-TES and TEOS (molar ratio: 22.5%/7.5%/70%, method B).

Microspheres with loading capacity of 20 wt % (Table 5) uracil were prepared using the procedure described in Example 3 with the exception that uracil powder was suspended in dispersed phase B2 before emulsification step; cyclohexane was used here as continuous phase. The obtained microspheres have an average particle size of 48 μm (FIG. 6-B). After the extraction of the active (i.e. uracil), the porosity data of the resulted microspheres are summarized in Table 5.

EXAMPLE 21-3: Preparation of uracil containing microspheres with fully hydrolyzed and pre-condensed C1-TES, C8-TES and TEOS (molar ratio: 22.5%/7.5%/70%, method B), as well as the non-hydrolyzed and non pre-condensed TMAPS.

Microspheres with loading capacity of 48 wt % (Table 5) uracil were prepared using the procedure described in Example 3 with the exception that 1) uracil powder was suspended in the dispersed phase (B2) before emulsification step, 2) cyclohexane was used here as continuous phase, and 3) after the overnight aging step of the suspension of microspheres, 10 mL of non-hydrolyzed and non pre-condensed TMAPS (50% in methanol) was added and the mixture was kept for another overnight. The average particles size of the obtained microspheres is 40 μm (FIG. 6-C). After the extraction of the active (i.e. uracil), the porosity data of the resulted microspheres are summarized in Table 5. Once suspended in water (pH=6), positively charged microspheres were generated with a zeta potential value of +50 mV. This confirms that TMAPS molecules are localized at the external outer surface of the microspheres.

EXAMPLE 21-4: Preparation of uracil containing microspheres with fully hydrolyzed and pre-condensed C1-TES, C8-TES and TEOS (molar ratio: 22.5%/7.5%/70%, method B), as well as the non-hydrolyzed and non pre-condensed DOAPS.

Microspheres with loading capacity of 46 wt % (Table 5) uracil were prepared using the procedure described in Example 3 with the exception that 1) uracil powder was suspended in the dispersed phase (B2) before emulsification step, 2) cyclohexane was used here as continuous phase, and 3) after the overnight aging step of the suspension of microspheres, 11 mL of non-hydrolyzed and non pre-condensed DOAPS (60% in ethanol) was added and the mixture was kept for another overnight. The resulted microspheres have an average particle size of 35 μm (FIG. 6-D). After the extraction of the active (i.e. uracil), the porosity data of the resulted microspheres are summarized in Table 5. Once suspended in water (pH=6), positively charged microspheres were generated with a zeta potential value of +55 mV. This confirms the presence DOAPS molecules at the external outer surface of the microspheres.

TABLE 5
Morphological and textural characteristics of the resulted microspheres:

| | Active loading | | | Porosity | | |
|---|---|---|---|---|---|---|
| | performance | | Particle | Surface | Pore | Average |
| | Loading | Sequestration | size | area | volume | pore size |
| Example | (wt %) | yield % | (d50, μm) | (m² · g⁻¹) | (cm³ · g⁻¹) | (nm) |
| Example 21-1 | 20 | 100 | 21 | 421 | 0.73 | 7.1 |
| Example 21-2 | 20 | 100 | 48 | 540 | 0.55 | 4.5 |
| Example 21-3 | 48 | 95 | 40 | 420 | 0.24 | 2.7 |
| Example 21-4 | 46 | 100 | 35 | 435 | 0.29 | 3.0 |

EXAMPLE 22: Preparation of active/payload charged microspheres by the procedure of adding the active/payload in the continuous phase; active/payload sequestrated at solid state (B4).

EXAMPLE 22-1: Preparation of uracil charged microspheres with fully hydrolyzed and non pre-condensed C18-TES, C8-TES and TEOS (molar ratio: 5%/5%/90%, method B).

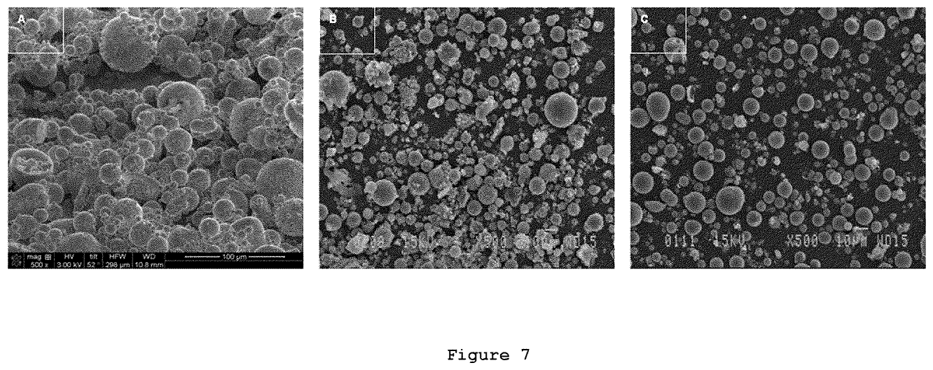

Microspheres with loading capacity of 20 wt % (Table 6) uracil were prepared using the procedure described in Example 1.1, except that uracil powder was suspended in the continuous phase (i.e. toluene) (B4). The obtained microspheres have an average size of 23 μm (FIG. 7-A). After the extraction of the active (i.e. uracil), the porosity data of the obtained microspheres are summarized in Table 6.

EXAMPLE 22-2: Preparation of 5-FU charged microspheres with fully hydrolyzed and non pre-condensed C18-TES and TEOS (molar ratio: 5%/95%, method B).

Microspheres with loading capacity of 13 wt % (Table 6) 5-FU were prepared using the procedure described in Example 1.1, except that 5-FU powder was suspended in the continuous phase (i.e. toluene) (B4). The obtained microspheres have an average size of 14 μm (FIG. 7-B). After the extraction of the active (i.e. 5-FU), the porosity data of the obtained microspheres are summarized in Table 6.

EXAMPLE 22-3: Preparation of 5-FU charged microspheres with fully hydrolyzed and non pre-condensed DOAPS and TEOS (molar ratio: 5%/95%, method B).

Microspheres containing 20 wt % (Table 6) 5-FU were prepared using the procedure described in Example 1.1, except that 5-FU powder was suspended in the continuous phase (i.e. toluene) (B4). The obtained microspheres have an average size of 14 μm (FIG. 7-C). After the extraction of the active (i.e. 5-FU), the porosity data of the obtained microspheres are summarized in Table 6.

TABLE 6
Morphological and textural characteristics of the resulted microspheres:

| | Active loading | | | Porosity | | |
|---|---|---|---|---|---|---|
| | performance | | Particle | Surface | Pore | Average |
| | Loading | Sequestration | size | area | volume | pore size |
| Example | (wt %) | yield (%) | (d50, μm) | (m² · g⁻¹) | (cm³ · g⁻¹) | (nm) |
| Example 22-1 | 20 | 99 | 23 | 350 | 0.55 | 9.0 |
| Example 22-2 | 13 | 100 | 14 | 520 | 0.50 | 3.5 |
| Example 22-3 | 19 | 99 | 15 | 432 | 0.50 | 3.5 |

EXAMPLE 23: Preparation of active/payload charged microspheres by the procedure of adding the active/payload into the emulsion; active/payload is sequestrated at solubilized state in water miscible solvent (B3).

EXAMPLE 23-1: Preparation of uracil charged microspheres with fully hydrolyzed and non pre-condensed C18-TES, C8-TES and TEOS (molar ratio: 5%/5%/90%, method B).

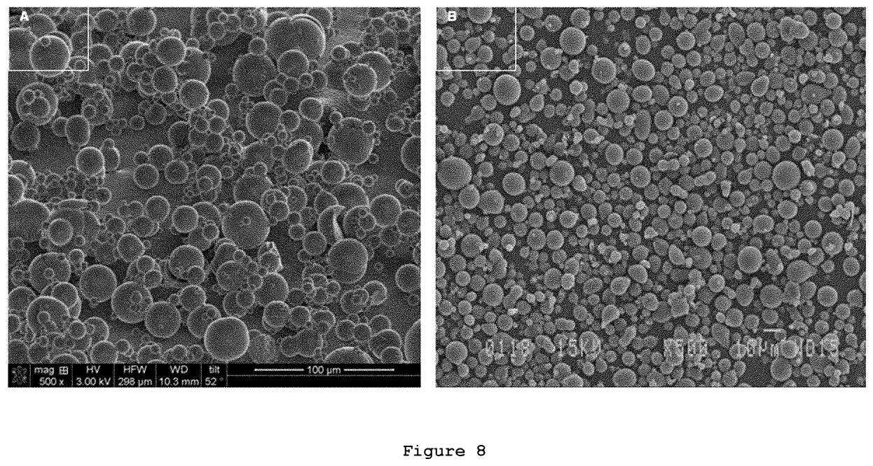

Microspheres with loading capacity of 5 wt % (Table 7) uracil were prepared using the procedure described in Example 1.1, except that uracil was solubilized in DMSO and added into the emulsion, after the emulsification step and before the adding of the condensation catalyst (B3); xylene was used here as the continuous phase. The average particles size of the resulted microspheres is 16 μm (FIG. 8-A). After the extraction of the active (i.e. uracil), the porosity data of the obtained microspheres are summarized in Table 7.

EXAMPLE 23-2: Preparation of uracil charged microspheres with fully hydrolyzed and non pre-condensed DOAPS and TEOS (molar ratio: 3%/97%, method B).

Microspheres with loading capacity of 5 wt % (Table 7) uracil were prepared using the procedure described in Example 1.1, except that uracil was solubilized in DMSO and added into the emulsion, after the emulsification step and before the adding of the condensation catalyst (B3); xylene was used here as the continuous phase. The average particles size of the resulted microspheres is 7 μm (FIG. 8-B). After the extraction of the active (i.e. uracil), the porosity data of the obtained microspheres are summarized in Table 7.

TABLE 7
Morphological and textural characteristics of the resulted microspheres:

| | Active loading | | | Porosity | | |
|---|---|---|---|---|---|---|
| | performance | | Particle | Surface | Pore | Average |
| | Loading | Sequestration | size | area | volume | pore size |
| Example | (wt %) | yield (%) | (d50, μm) | (m² · g⁻¹) | (cm³ · g⁻¹) | (nm) |
| Example 23-1 | 5 | 100 | 16 | 674 | 1.09 | 6.4 |
| Example 23-2 | 5 | 99 | 7 | 456 | 0.91 | 8.5 |

EXAMPLE 24: Preparation of active/payload charged microspheres by the procedure of adding the active/payload into the emulsion; active/payload is sequestrated at solubilized state in the condensation catalyst (B5).

EXAMPLE 24-1: Preparation of uracil charged microspheres with fully hydrolyzed and pre-condensed C1-TES, C8-TES and TEOS (molar ratio: 22.5%/7.5/70%, method B).

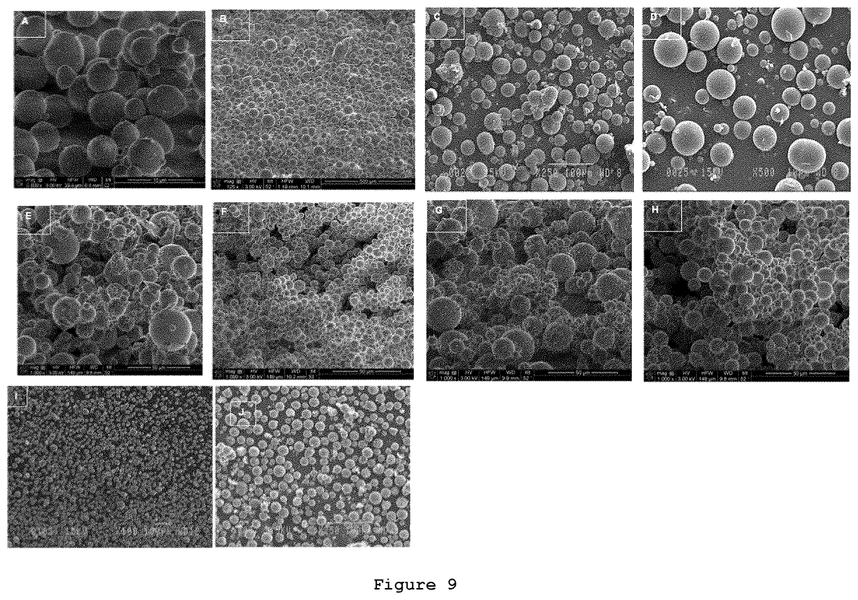

Microspheres with loading capacity of 1 wt % uracil were prepared using the procedure described in Example 3, except that uracil was solubilized in the used condensation catalyst (i.e. NaOH was used here) (B5); cyclohexane was used here as the continuous phase. The average particles size of the resulted microspheres is 9 μm (FIG. 9-A).

EXAMPLE 24-2: Preparation of uracil charged microspheres with fully hydrolyzed and non pre-condensed C4-TES and TEOS (molar ratio: 35%/65%, method B).

Microspheres with loading capacity of 4 wt % (Table 8) uracil are prepared using the procedure described in Example 1.1, except that uracil was solubilized in the condensation catalyst solution (i.e. NH₄OH was added here) (B5). The average particles size of the resulted microspheres is 34 μm (FIG. 9-B). After the extraction of the active (i.e. uracil), the porosity data of the obtained microspheres are summarized in Table 8.

EXAMPLE 24-3: Preparation of uracil charged microspheres with fully hydrolyzed and non pre-condensed C8-TES and TEOS (molar ratio: 10%/90%, method B).

Microspheres with loading capacity of 4 wt % (Table 8) uracil were prepared using the procedure described in Example 1.1, except that uracil was solubilized in the condensation catalyst solution (i.e. NH₄OH was added here) (B5). The average particles size of the resulted microspheres is 14 μm (FIG. 9-C). After the extraction of the active (i.e. uracil), the porosity data of the obtained microspheres are summarized in Table 8.

EXAMPLE 24-4: Preparation of uracil containing microspheres with fully hydrolyzed and non pre-condensed C18-TES, C8-TES and TEOS (molar ratio: 5%/5%/90%, method B).

Microspheres with loading capacity of 2 wt % (Table 8) uracil were prepared using the procedure described in Example 1.1, except that uracil was solubilized in the condensation catalyst solution (i.e. NH₄OH was added here) (B5). The average particles size of the resulted microspheres is 21 μm (FIG. 9-D). After the extraction of the active (i.e. uracil), the porosity data of the obtained microspheres are summarized in Table 8.

EXAMPLE 24-5: Preparation of uracil containing microspheres with fully hydrolyzed and non pre-condensed C18-TES, C8-TES and TEOS (molar ratio: 10%/10%/90%, method B).

Microspheres with loading capacity of 2 wt % (Table 8) uracil were prepared using the procedure described in Example 1.1, except that uracil was solubilized in the condensation catalyst solution (i.e. NH₄OH was added here) (B5). The average particles size of the resulted microspheres is 21 μm (FIG. 9-E). After the extraction of the active (i.e. uracil), the porosity data of the obtained microspheres are summarized in Table 8.

EXAMPLE 24-6: Preparation of uracil containing microspheres with fully hydrolyzed and non pre-condensed DOAPS and TEOS (molar ratio: 3%/97%, method B).

Microspheres with loading capacity of 2 wt % (Table 8) uracil are prepared using the procedure described in Example 1.1, except that uracil was solubilized in the condensation catalyst solution (i.e. NH₄OH was added here) (B5). The average particles size of the resulted microspheres is 5 μm (FIG. 9-F). After the extraction of the active (i.e. uracil), the porosity data of the obtained microspheres are summarized in Table 8. Once suspended in water (pH=6), positively charged microspheres were generated with a zeta potential value of +55 mV. This demonstrates the presence of DOAPS molecules at the external outer surface of the microspheres.

EXAMPLE 24-7: Preparation of uracil containing microspheres with fully hydrolyzed and non pre-condensed DOAPS, C8-TES and TEOS (molar ratio: 3%/5%/93%, method B).

Microspheres with loading capacity of 2 wt % (Table 8) uracil were prepared using the procedure described in Example 1.1, except that uracil was solubilized in the condensation catalyst solution (i.e. NH₄OH was added here) (B5). The average particles size of the resulted microspheres is 12 μm (FIG. 9-G). After the extraction of the active (i.e. uracil), the porosity data of the obtained microspheres are summarized in Table 8. Once suspended in water (pH=6), positively charged microspheres were generated with a zeta potential value of +10 mV. This demonstrates the presence of DOAPS molecules at the external outer surface of the microspheres.

EXAMPLE 24-8: Preparation of uracil containing microspheres with fully hydrolyzed and non pre-condensed DOAPS, C18-TES and TEOS (molar ratio: 3%/5%/93%, method B).

Microspheres with loading capacity of 2 wt % (Table 8) uracil were prepared using the procedure described in Example 1.1, except that uracil was solubilized in the condensation catalyst solution (i.e. NH₄OH was added here) (B5). The average particles size of the resulted microspheres is 18 μm (FIG. 9-H). After the extraction of the active (i.e. uracil), the porosity data of the obtained microspheres are summarized in Table 8. Once suspended in water (pH=6), positively charged microspheres were generated with a zeta potential value of +29 mV. This demonstrates the presence of DOAPS molecules at the external outer surface of the microspheres.

EXAMPLE 24-9: Preparation of 5-FU containing microspheres with fully hydrolyzed and non pre-condensed C18-TES and TEOS (molar ratio: 5%/95%, method B).

Microspheres with loading capacity of 7 wt % (Table 8) 5-FU were prepared using the procedure described in Example 1.1, except that uracil was solubilized in the condensation catalyst solution (i.e. hot NH₄OH is added here) (B5). The average particles size of the resulted microspheres is 15 μm (FIG. 9-I). After the extraction of the active (i.e. 5-FU), the porosity data of the obtained microspheres are summarized in Table 8.

EXAMPLE 24-10: Preparation of uracil containing microspheres with fully hydrolyzed and non pre-condensed 100% TEOS (method B).

Microspheres with loading capacity of 5 wt % (Table 8) uracil were prepared using the procedure described in Example 1.1, except that uracil was solubilized in the condensation catalyst solution (i.e. hot NH₄OH was added here) (B5). The average particles size of the resulted microspheres is 27 μm (FIG. 9-J). After the extraction of the active (i.e. uracil), the porosity data of the obtained microspheres are summarized in Table 8.

The porosity data of microspheres that initially contain an active/payload is always higher than that of the corresponding microspheres without the presence of active/payload.

TABLE 8
Morphological and textural characteristics of the resulted microspheres:

| | Active loading | | | Porosity | | |
|---|---|---|---|---|---|---|
| | performance | | Particle | Surface | Pore | Average |
| | Loading | Sequestration | size | area | volume | pore size |
| Example | (wt %) | yield (%) | (d50, μm) | (m² · g⁻¹) | (cm³ · g⁻¹) | (nm) |
| Example 24-2 | 4 | 80 | 34 | 68 | 0.10 | 8.0 |
| Example 24-3 | 4 | 90 | 14 | 115 | 0.28 | 8.5 |
| Example 24-4 | 2 | 80 | 21 | 133 | 0.30 | 8.1 |
| Example 25-5 | 1 | 70 | 13 | 70 | 0.09 | 7.9 |
| Example 24-6 | 2 | 75 | 5 | 220 | 0.69 | 10.2 |
| Example 24-7 | 2 | 77 | 12 | 261 | 0.50 | 6.1 |
| Example 24-8 | 2 | 70 | 18 | 261 | 0.71 | 10.1 |
| Example 24-9 | 7 | 95 | 15 | 330 | 0.51 | 6.3 |
| Example 24-10 | 5 | 75 | 27 | 605 | 0.99 | 6.5 |

EXAMPLE 25: Preparation of active/payload charged microspheres by the combination of two or more active/payload adding strategy: e.g. adding active/payload at solubilized state in both the dispersed phase and the condensation catalyst solution.

Preparation of uracil charged microspheres with fully hydrolyzed and pre-condensed C1-TES, C8-TES and TEOS (molar ratio: 22.5%/7.5/70%, method B). Microspheres with loading capacity of 10 wt % uracil are prepared using the procedure described in Example 3, except that 1) Uracil was solubilized in dispersed phase (B2) and also 2) uracil was solubilized in condensation catalyst solution (i.e. NH₄OH) (B5); cyclohexane was used here as the continuous phase. The textural and structural properties of the obtained microspheres are summarized in FIG. 9-K and Table 9.

TABLE 9
Textural and structural properties of the obtained microspheres

| | Active loading | | | Porosity | | |
|---|---|---|---|---|---|---|
| | performance | | Average | Surface | Pore | Pore |
| | Loading | Sequestration | particle | area | volume | size |
| Example | (wt %) | yield (%) | size (μm) | (m² · g⁻¹) | (cm³ · g⁻¹) | (nm) |
| Example 25 | 10 | 100 | 46 | 433 | 0.30 | 2.9 |

EXAMPLE 26: Examples of the controlled release performances achieved with the obtained microspheres.

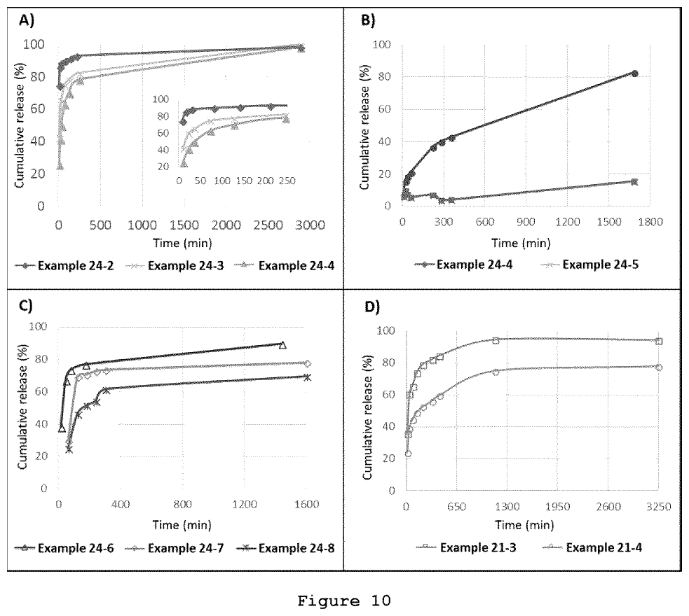

The controlled of active/payload release can be achieved in function of the hydrophobicity/hydrophilicity of the matrix and the external surface as shown in FIG. 10.

Interactions between the payloads/actives and the surface have a major impact on the kinetics release. External surface of the spheres can play the role of a diffusion barrier which significantly affects the active release rate. The barrier can be the consequence of a repulsive interaction from the external layer (in our case hydrophilic/hydrophobic repulsion) or/and a steric confinement (long-chain organosilane). The more hydrophobic groups are in the matrix, the slower the release is. It has been shown that after one hour, depending of the matrix composition, 5 to 80% of the initial quantity of actives/payloads present in the microspheres was released. Similar results are obtained with different active content loadings. Therefore, the choice of the organosilane for the microspheres matrix is highly significant for tuning actives/payloads release kinetics.

### Sample Characterization

Specific surface area (BET) and porosity: The surface area and porosity of the silica microspheres are characterized with Micrometrics TriStar™ 3000 V4.01 and Micrometrics TriStar™ 3020 V3.02 at 77 K. The collected data are analyzed using the standard Brunauer-Emmett-Teller (BET) to get the surface area, and the pore size is obtained from the maxima of the pore size distribution curve calculated by Barrett-Joyner-Halenda (BJH) method using the adsorption branch of the isotherm.

Particles size distribution: To measure the particle size distribution, Silica nano-/microspheres (about 50 mg) is dispersed in methanol of about 5 mL in ultrasonic bath for 5 minutes to obtain a well dispersed solution, which is then added into the sonicated bath of Malvern Mastersizer 2000 (Hydro 2000S, Model AWA2001) till the obstruction of the signal is about 5 to 8%.

Active Quantification in Silica Sphere: The loading of actives sequestered silica spheres are determined by suspending certain amount of sequestered silica spheres containing about 100 mg actives in 10 mL of a 10% ammonia aqueous solution, which is then sonicated in Branson 8800 ultrasonic bath for 30 minutes, and followed by 2 hours shaking with using IKA HS-501 Horizontal shaker at 200 mot/min to achieve fully release. The silica spheres are filtered off through a 0.22 μm filter to give a clear solution for HPLC analysis.

The HPLC used to determine the active concentration of the solution obtained above is Agilent 1100 equipped with a quaternary solvent delivery system (G1311A), vacuum degasser unit (G1322A), UV photodiode array detector (G1314A), standard autosampler (G1313A) and thermostatic column compartment (G1316A)). The SiliaChrom DtC18 column of 3×150 mm i.d., 5 μm, 100 Å, is used to detect the actives. 0.1% formic acid containing water is used as the mobile phase MPA while the mobile phase MPB is 0.1% formic acid containing acetonitrile. The injections volume is 2 μL. The Starting mobile phase is 95% MPA and 5% MPB, and ends at 95% MPB at 4 minutes, hold for another 2 minutes. The flow rate, column temperature and the detector are set at 0.5 ml/min, 23° C. and 260 nm respectively. Uracil retention time is 1.88 min, and 5-FU retention time is 2.39 min. The calibration curves are constructed with pure compounds purchased from Sigma Aldrich.

Scanning Electron Microscopy (SEM): SEM images of the microspheres are recorded with FEI Quanta-3D-FEG at 3.0 kV without coating or with JEOL 840-A at 15 kV with gold coating.

Water Quantification in Silica (Karl Fisher): The water percentage is estimated by using titrator Compact V20s from Mettler Toledo.

Zeta potential: To determine the Zeta potential of the nano-/microspheres, the suspension is first prepared by dispersing 10 mg of nano-/microspheres in water of 10 mL and followed by sonication for 10 min and vortex for 1 min. The mixture is further diluted 10 times and placed in a Capillary Zeta Cell for the zeta potential measurement with Malvern, Zetasizer Nano ZS.

Contact angle: A few milligrams of nano-/microspheres are deposited on one side of a Micro-Tec D12 double sided non-conductive adhesive, which is fixed on to a Microscope glass slide. The sample layer is smoothed as much as possible. The contact angle is then characterized with VCA 2500 XE system.

Elemental analysis (CNS and ICP-ES): Carbon, nitrogen and sulfur contents are measured with Perkin Elmer 2400 Series II CHNS/O Analyzer. Silicon content is measured with ICP-ES.

X-ray Photoelectron Spectroscopy (XPS): The chemical composition of the external surface was investigated in a maximum depth of 5 nanometers by X-ray photoelectron spectroscopy, using Axis-Ultra de Kratos (UK). The main XPS chamber was maintained at a base pressure of <5.10⁻⁸ Torr. A monochromatic aluminum X-ray source (Al kα=1486.6 eV) at 250W was used to record survey spectra (1400-0 eV) and high-resolution spectra with charge neutralization. The detection angle was set at 45° with respect to the normal of the surface and the analyzed area was 0.016 cm² (aperture 5).

Thermogravimetric analysis-differential scanning calorimetry analysis (TGA-DSC): Measurements were performed using a Netzsch STA 449C thermogravimetric analyzer, under an airflow rate of 20 mL.min⁻¹, with a heating rate of 10° C.min⁻¹, between 35 and 700° C.

1. A process of preparation of organosiloxane nano-/microspheres comprising:
i0) separately hydrolyzing one or more silica precursor in a hydrolytic media to provide one or more pre-hydrolyzed silica precursor;
i1) combining the pre-hydrolyzed silica precursors of step i0) to provide a dispersed phase comprising combined pre-hydrolyzed silica precursors; or
i2) removing a part or totality of volatile solvents from said combined pre-hydrolyzed silica precursors to provide a dispersed phase comprising pre-condensed silica precursors; or
i3) preparing a dispersed phase comprising a hydrophilic solvent by adding said hydrophilic solvent to said dispersed phase comprising combined pre-hydrolyzed silica precursors obtained in step i1) or by adding said hydrophilic solvent to said dispersed phase comprising pre-condensed silica precursors obtained in step i2);
i4) emulsifying, in absence of a surfactant, the dispersed phase of the step i1), i2) or i3) in a continuous phase to provide a water in oil emulsion;
i5) adding a condensation catalyst to the emulsion of step i4) to provide said organosiloxane nano-/microspheres.

2. The process of claim 1, wherein the silica precursor has the formula R₄₋ₓSi(L)ₓ or formula (L)₃Si—R′—Si(L)₃, wherein:
R: is mono-silylated residue as an alkyl, alkenyl, alkynyl, alicyclic, aryl, alkyl-aryl group, which is optionally substituted by a halogen atom, glycidyloxy-, —OH, —SH, polyethylene glycol (PEG), —N(Rₐ)₂, —N⁺(Rₐ)₃;
L: is a halogen or an acetoxide —O—C(O)Rₐ, or alkoxide ORₐ group;
R′: is bi-silylated residue as an alkyl, alkenyl, alkynyl, alicyclic, aryl, alkyl-aryl group, which is optionally substituted by a halogen atom, —OH, —SH, —N(Rₐ)₂, —N⁺(Rₐ)₃;
Rₐ: can be hydrogen, alkyl, alkenyl, alkynyl, alicyclic, aryl and alkyl-aryl; and
X: is an integer of 1 to 4 or alternatively x is an integer of 1 to 3.

3. The process of claim 1, wherein an active/payload insoluble in the continuous phase is added at step (i1) in the combined pre-hydrolyzed silica precursor.

4. The process of claim 1, wherein an active/payload insoluble in the continuous phase is added at step (i2) in the pre-condensed silica precursor.

5. The process of claim 1, wherein an active/payload insoluble in the continuous phase is added at step (i3) in the dispersed phase.

6. The process of claim 1, wherein an active/payload insoluble in the continuous phase is added at step (i4) in the continuous phase.

7. The process of claim 1, wherein an active/payload insoluble in the continuous phase is added at step (i4) in the emulsion.

8. The process of claim 1, wherein an active/payload insoluble in the continuous phase is added at step (i5) in the condensation catalyst.

9. The process of claim 1, wherein said active/payload insoluble in the continuous phase, is a hydrophilic molecule in a liquid state.

10. The process of claim 1, wherein said active/payload insoluble in the continuous phase, is a hydrophilic molecule in a solid state.

11. The process of claim 1, wherein said active/payload insoluble in the continuous phase, is a cosmetic, cosmeceutical or pharmaceutical compound.

12. The process of claim 1, wherein said active/payload insoluble in the continuous phase, is 5-fluorouracil.

13. The process of claim 1, wherein said active/payload insoluble in the continuous, is a saccharide or a derivative.

14. An organosiloxane spheroidal nano-/microspheres prepared by the process as defined in claim 1 comprising a network consisting of organo-siloxane, wherein said particle is uncalcined, amorphous, surfactant-free and is sub-micron to micron size, particle optionally comprising an active/payload.

15. The organosiloxane spheroidal nano-/microspheres as defined in claim 14,
wherein said organosiloxane spheroidal nano-/microspheres are sub-micron to micron size;
wherein said organosiloxane spheroidal nano-/microsphere are porous as assessed by pore volume, pore diameter and specific surface area as measured by N₂ physisorption;
wherein the external surface hydrophobic/hydrophilic property of the organosiloxane spheroidal nano-/microspheres, assessed by contact angle measurement is hydrophilic if said contact angle is inferior to 90°, or is hydrophobic if said contact angle is superior to 900 or has a balanced hydrophobicity if said contact angle is from 850 to 95°.

16-17. (canceled)

18. A method for modulating the release of an active/payload, comprising incorporating said active/payload by a process as defined in claim 1.
